(19) United States
(12) Patent Application Publication
(10) Pub. No.: US 2026/0000515 A1
(43) Pub. Date: Jan. 1, 2026

(54) ACETABULAR PROSTHESIS SYSTEMS AND SURGICAL METHODS

(71) Applicant: George J. HAIDUKEWYCH, Orlando, FL (US)

(72) Inventor: George J. HAIDUKEWYCH, Orlando, FL (US)

(21) Appl. No.: 18/755,870

(22) Filed: Jun. 27, 2024

(51) Int. Cl. A61F 2/34; A61F 2/30

(52) U.S. Cl. CPC A61F 2/34; A61F 2002/30121; A61F 2002/30774; A61F 2002/30787; A61F 2002/3403

(57) ABSTRACT

An acetabular cub system is configured to be secured to a pelvis and may include an acetabular cup and a plurality of bone fasteners. The acetabular cup may include a bone engaging surface and an articulating surface, as well as a plurality of lobed fastener apertures extending from a first opening at the articulating surface to a second opening at the bone engaging surface. The plurality of bone fasteners may be insertable through the lobed fastener apertures to secure the acetabular cup to the pelvis. Each of the plurality of lobed fastener apertures may include a central aperture configured to receive a first bone fastener of the plurality of bone fasteners and one or more aperture lobes intersecting with the central aperture configured to receive a second bone fastener of the plurality of bone fasteners. The central aperture may be larger than the one or more aperture lobes.

### TECHNICAL FIELD

The present disclosure relates to systems and methods for use in orthopedic surgery. More specifically, the present disclosure relates to acetabular prothesis systems and surgical methods.

### BACKGROUND

Total hip arthroplasty is a widely performed surgical procedure aimed at alleviating pain and restoring mobility in patients suffering from hip joint degeneration or trauma. Central to the success of hip replacement surgery is the secure fixation of the acetabular cup, the component that replaces the damaged hip socket. Acetabular cups typically use screws to attach the device to the bone, however, conventional screw configurations often encounter challenges in achieving optimal stability and longevity, particularly in cases of poor bone quality, anatomical variations, or the need for precise screw placement to address patient-specific requirements. Recognizing the limitations of existing fixation techniques, there is a need for innovation in acetabular cup design to enhance fixation stability and surgical outcomes.

### SUMMARY

The various systems and methods of the present disclosure have been developed in response to the present state of the art, and in particular, in response to the problems and needs in the art that have not yet been fully solved by currently available acetabular prosthetic systems and methods.

In some embodiments, an acetabular cub system configured to be secured to a pelvis may include an acetabular cup and a plurality of bone fasteners. The acetabular cup may include a bone engaging surface and an articulating surface, as well as a plurality of lobed fastener apertures extending from a first opening at the articulating surface to a second opening at the bone engaging surface. The plurality of bone fasteners may be insertable through the lobed fastener apertures to secure the acetabular cup to the pelvis. Each of the plurality of lobed fastener apertures may include a central aperture configured to receive a first bone fastener of the plurality of bone fasteners and one or more aperture lobes intersecting with the central aperture configured to receive a second bone fastener of the plurality of bone fasteners. The central aperture may be larger than the one or more aperture lobes.

In the acetabular cup system of any preceding paragraph, each of the lobed fastener apertures may be configured so that, with the first bone fastener received in the central aperture and anchored in the pelvis, and the second bone fastener received in one of the one or more aperture lobes and anchored in the pelvis, the second bone fastener inhibits the first bone fastener from disengaging from the pelvis.

In the acetabular cup system of any preceding paragraph, the first bone fastener may include a first fastener head and the second bone fastener may include a second fastener head. The lobed fastener apertures may be shaped such that the first fastener head and the second fastener head seat at different depths within the lobed fastener apertures.

In the acetabular cup system of any preceding paragraph, the second fastener head may overlap the first fastener head.

In the acetabular cup system of any preceding paragraph, the first bone fastener may be longer than the second bone fastener.

In the acetabular cup system of any preceding paragraph, the acetabular cup system may include two aperture lobes and three bone fasteners. Each of the two aperture lobes and the central aperture may be configured to receive one of the three bone fasteners.

In the acetabular cup system of any preceding paragraph, the first bone fastener may include a first proximal end configured to engage the acetabular cup and a first distal end configured to be anchored within the pelvis. The second bone fastener may include a second proximal end configured to engage the acetabular cup and a second distal end configured to be anchored within the pelvis, and the lobed fastener apertures are shaped such that the first proximal end and the second proximal end are adjacent, and the first distal end and the second distal end diverge within the pelvis.

In the acetabular cup system of any preceding paragraph, the first bone fastener may include a first fastener head including a first thread, the second bone fastener may include a second fastener head including a second thread, and at least one of the central aperture and the one or more aperture lobes may include a third thread. The third thread may correspond to at least one of the first thread and the second thread.

In some embodiments, an acetabular cup system may be configured to be secured to a pelvis. The acetabular cup system may include an acetabular cup including a bone engaging surface, and articulating surface, and a plurality of lobed fastener apertures extending from a first opening at the articulating surface to a second opening at the bone engaging surface. The acetabular cup system may also include a plurality of bone fasteners insertable through the lobed fastener apertures to secure the acetabular cup to the pelvis. Each of the plurality of fastener apertures may include a first aperture configured to receive a first bone fastener of the plurality of bone fasteners, a second aperture intersecting with the first aperture and configured to receive a second bone fastener of the plurality of bone fasteners. The first aperture and the second aperture may be generally equally sized.

In the acetabular cup system of any preceding paragraph, the acetabular cup may be further configured so that, with the first bone fastener received in the first aperture and anchored in the pelvis, and the second bone fastener received in the second aperture and anchored in the pelvis, the second bone fastener inhibits the first bone fastener from disengaging from the pelvis.

In the acetabular cup system of any preceding paragraph, the first bone fastener may include a first fastener head. The second bone fastener may include a second fastener head, and the fastener apertures may be shaped such that the first fastener head and the second fastener head seat at different depths within the fastener apertures.

In the acetabular cup system of any preceding paragraph, the second fastener head may overlap the first fastener head.

In the acetabular cup system of any preceding paragraph, the first bone fastener may include a first proximal end configured to engage the acetabular cup and a first distal end configured to be anchored within the pelvis. The second bone fastener may include a second proximal end configured to engage the acetabular cup and a second distal end configured to be anchored within the pelvis and the fastener apertures may be shaped such that the first proximal end and the second proximal end are adjacent, and the first distal end and the second distal end diverge within the pelvis.

In the acetabular cup system of any preceding paragraph, the first bone fastener may include a first fastener head including a first thread. The second bone fastener may include a second fastener head including a second thread, and at least one of the first aperture and the second aperture may include a third thread. The third thread may correspond to at least one of the first thread and the second thread.

In some embodiments, an acetabular cup system may be configured to be secured to a pelvis. The acetabular cup system may include an acetabular cup including a bone engaging surface, and articulating surface, and a plurality of fastener apertures. The acetabular cup system may also include a plurality of bone fasteners insertable through the lobed fastener apertures to secure the acetabular cup to the pelvis. Each of the plurality of fastener apertures may include a central aperture extending from a first opening at the articulating surface to a second opening at the bone engaging surface and configured to receive a first bone fastener of the plurality of bone fasteners, and at least one aperture lobe extending from a third opening at the articulating surface to a fourth opening at the bone engaging surface and configured to receive a second bone fastener of the plurality of bone fasteners. The first opening and the third opening intersect, and the second opening and the fourth opening do not intersect.

In the acetabular cup system of any preceding paragraph, the first bone fastener may include a first fastener head, and the second bone fastener may include a second fastener head. The acetabular cup may be further configured so that, with the first bone fastener received in the central aperture and anchored in the pelvis, and the second bone fastener received in the aperture lobe and anchored in the pelvis, the first fastener head is not in contact with the second fastener head.

In the acetabular cup system of any preceding paragraph, the fastener apertures may be shaped such that the first fastener head and the second fastener head seat at different depths within the fastener apertures.

In the acetabular cup system of any preceding paragraph, the first bone fastener may be longer than the second bone fastener.

In the acetabular cup system of any preceding paragraph, the first bone fastener may include a first proximal end configured to engage the acetabular cup and a first distal end configured to be anchored within the pelvis, the second bone fastener including a second proximal end configured to engage the acetabular cup and a second distal end configured to be anchored within the pelvis. The fastener apertures may be shaped such that the first proximal end and the second proximal end are adjacent and the first distal end and the second distal diverge within the pelvis.

In the acetabular cup system of any preceding paragraph, the first bone fastener may include a first fastener head including a first thread. The second bone fastener may include a second fastener head including a second thread, and at least one of the central aperture and the aperture lobe may include a third thread. The third thread may correspond to at least one of the first thread and the second thread.

These and other features and advantages of the present disclosure will become more fully apparent from the following description and appended claims or may be learned by the practice of the implants, systems, and methods set forth hereinafter.

### BRIEF DESCRIPTION OF THE DRAWINGS

Exemplary embodiments of the present disclosure will become more fully apparent from the following description taken in conjunction with the accompanying drawings. Understanding that these drawings depict only exemplary embodiments and are, therefore, not to be considered limiting of the scope of the present disclosure, the exemplary embodiments of the present disclosure will be described with additional specificity and detail through use of the accompanying drawings.

FIG. 1A is a side view of an acetabular cup according to an embodiment of the present disclosure.

FIG. 1B is a front section view of the acetabular cup of FIG. 1A.

FIG. 1C is a perspective view of the acetabular cup of FIG. 1A.

FIG. 2A is a side view of an acetabular cup and fasteners according to an embodiment of the present disclosure.

FIG. 2B is a front section view of the acetabular cup and the fasteners of FIG. 2A.

FIG. 2C is a perspective view of the acetabular cup and the fasteners of FIG. 2A.

FIG. 3A is a side view of an acetabular cup according to an embodiment of the present disclosure.

FIG. 3B is a front section view of the acetabular cup of FIG. 3A.

FIG. 3C is a perspective view of the acetabular cup of FIG. 3A.

FIG. 4A is a side view of an acetabular cup and fasteners according to an embodiment of the present disclosure.

FIG. 4B is a front section view of the acetabular cup and the fasteners of FIG. 4A.

FIG. 4C is a perspective view of the acetabular cup and the fasteners of FIG. 4A.

FIG. 5A is a side view of an acetabular cup according to an embodiment of the present disclosure.

FIG. 5B is a front section view of the acetabular cup of FIG. 5A.

FIG. 5C is a perspective view of the acetabular cup of FIG. 5A.

FIG. 6A is a side view of an acetabular cup and fasteners according to an embodiment of the present disclosure.

FIG. 6B is a front section view of the acetabular cup and the fasteners of FIG. 6A.

FIG. 6C is a perspective view of the acetabular cup and the fasteners of FIG. 6A.

FIG. 7A is a front section view of an acetabular cup and fasteners according to an embodiment of the present disclosure.

FIG. 7B is a bottom detail view of the fasteners of FIG. 7A.

FIG. 8A is a side view of an acetabular cup according to an embodiment of the present disclosure.

FIG. 8B is a front section view of the acetabular cup of FIG. 8A.

FIG. 8C is a perspective view of the acetabular cup of FIG. 8A.

FIG. 9A is a side view of an acetabular cup and a fastener according to an embodiment of the present disclosure.

FIG. 9B is a front section view of the acetabular cup and the fastener of FIG. 9A.

FIG. 9C is a perspective view of the acetabular cup and the fastener of FIG. 9A.

FIG. 10A is a side view of an acetabular cup according to an embodiment of the present disclosure.

FIG. 10B is a front section view of the acetabular cup of FIG. 10A.

FIG. 10C is a perspective view of the acetabular cup of FIG. 10A.

FIG. 11A is a side view of an acetabular cup and fasteners according to an embodiment of the present disclosure.

FIG. 11B is a front section view of the acetabular cup and the fasteners of FIG. 11A.

FIG. 11C is a perspective view of the acetabular cup and the fasteners of FIG. 11A.

FIG. 12A is a bottom detail view of fasteners according to an embodiment of the present disclosure.

FIG. 12B is a front section view of an acetabular cup and the fasteners of FIG. 12A.

FIG. 13A is a side view of an acetabular cup according to an embodiment of the present disclosure.

FIG. 13B is a front section view of the acetabular cup of FIG. 13A.

FIG. 13C is a perspective view of the acetabular cup of FIG. 13A.

FIG. 14A is a side view of an acetabular cup and fasteners according to an embodiment of the present disclosure.

FIG. 14B is a front section view of the acetabular cup and the fasteners of FIG. 14A.

FIG. 14C is a perspective view of the acetabular cup and the fasteners of FIG. 14A.

FIG. 15A is a side view of an acetabular cup according to an embodiment of the present disclosure.

FIG. 15B is a front section view of the acetabular cup of FIG. 15A.

FIG. 15C is a perspective view of the acetabular cup of FIG. 15A.

FIG. 16A is a side view of an acetabular cup and fasteners according to an embodiment of the present disclosure.

FIG. 16B is a front section view of the acetabular cup and the fasteners of FIG. 16A.

FIG. 16C is a perspective view of the acetabular cup and the fasteners of FIG. 16A.

FIG. 17A is a side view of an acetabular cup according to an embodiment of the present disclosure.

FIG. 17B is a front section view of the acetabular cup of FIG. 17A.

FIG. 17C is a perspective view of the acetabular cup of FIG. 17A.

FIG. 18A is a side view of an acetabular cup and fasteners according to an embodiment of the present disclosure.

FIG. 18B is a front section view of the acetabular cup and the fasteners of FIG. 18A.

FIG. 18C is a perspective view of the acetabular cup and the fasteners of FIG. 18A.

FIG. 18D is a bottom view of the acetabular cup and the fasteners of FIG. 18A.

FIG. 19A is a front section view of an acetabular cup and a fastener according to an embodiment of the present disclosure.

FIG. 19B is a top detail view of an aperture of the acetabular cup of FIG. 19A.

FIG. 20A is a front section view of an acetabular cup and fasteners according to an embodiment of the present disclosure.

FIG. 20B is a top detail view of an aperture of the acetabular cup of FIG. 20A.

FIG. 21A is a front section view of an acetabular cup and fasteners according to an embodiment of the present disclosure.

FIG. 21B is a top detail view of an aperture of the acetabular cup of FIG. 21A.

FIG. 22A is a front section view of an acetabular cup and fasteners according to an embodiment of the present disclosure.

FIG. 22B is a top detail view of an aperture of the acetabular cup of FIG. 22A.

FIG. 23 is a perspective view of an acetabular cup with fasteners implanted in an exemplary pelvis according to an embodiment of the present disclosure.

FIG. 24 is a perspective view of an acetabular cup with fasteners implanted in an exemplary pelvis according to an embodiment of the present disclosure.

It is to be understood that the drawings are for purposes of illustrating the concepts of the present disclosure and may not be drawn to scale. Furthermore, the drawings illustrate exemplary embodiments and do not represent limitations to the scope of the present disclosure.

### DETAILED DESCRIPTION

Exemplary embodiments of the present disclosure will be best understood by reference to the drawings, wherein like parts are designated by like numerals throughout. It will be readily understood that the components of the present disclosure, as generally described and illustrated in the drawings, could be arranged, and designed in a wide variety of different configurations. Thus, the following more detailed description of the embodiments of the devices, systems, and methods, as represented in the drawings, is not intended to limit the scope of the present disclosure but is merely representative of exemplary embodiments of the present disclosure.

The word “exemplary” is used herein to mean “serving as an example, instance, or illustration.” Any embodiment described herein as “exemplary” is not necessarily to be construed as preferred or advantageous over other embodiments. While the various aspects of the embodiments are presented in the drawings, the drawings are not necessarily drawn to scale unless specifically indicated.

Standard medical planes of reference and descriptive terminology are employed in this specification. While these terms are commonly used to refer to the human body, certain terms are applicable to physical objects in general.

A standard system of three mutually perpendicular reference planes is employed. A sagittal plane divides a body into right and left portions. A coronal plane divides a body into anterior and posterior portions. A transverse plane divides a body into superior and inferior portions. A mid-sagittal, mid-coronal, or mid-transverse plane divides a body into equal portions, which may be bilaterally symmetric. The intersection of the sagittal and coronal planes defines a superior-inferior or cephalad-caudal axis. The intersection of the sagittal and transverse planes defines an anterior-posterior axis. The intersection of the coronal and transverse planes defines a medial-lateral axis. The superior-inferior or cephalad-caudal axis, the anterior-posterior axis, and the medial-lateral axis are mutually perpendicular.

Anterior means toward the front of a body. Posterior means toward the back of a body. Superior or cephalad means toward the head. Inferior or caudal means toward the feet or tail. Medial means toward the midline of a body, particularly toward a plane of bilateral symmetry of the body. Lateral means away from the midline of a body or away from a plane of bilateral symmetry of the body. Axial means toward a central axis of a body. Abaxial means away from a central axis of a body. Ipsilateral means on the same side of the body. Contralateral means on the opposite side of the body. Proximal means toward the trunk of the body. Proximal may also mean toward a user or operator. Distal means away from the trunk. Distal may also mean away from a user or operator. Dorsal means toward the top of the foot. Plantar means toward the sole of the foot. Varus means deviation of the distal part of the leg below the knee inward, resulting in a bowlegged appearance. Valgus means deviation of the distal part of the leg below the knee outward, resulting in a knock-kneed appearance.

The present disclosure relates to acetabular systems and surgical methods. Those skilled in the art will recognize that the following description is merely illustrative of the principles of the technology, which may be applied in various ways to provide many alternative embodiments. For the purposes of illustrating the concepts of the present design, the present disclosure describes acetabular systems and surgical methods to be used in hip replacement surgery. However, it will be understood that other variations and uses are contemplated, including, but not limited to, applications in the shoulder and/or other ball and socket type joints.

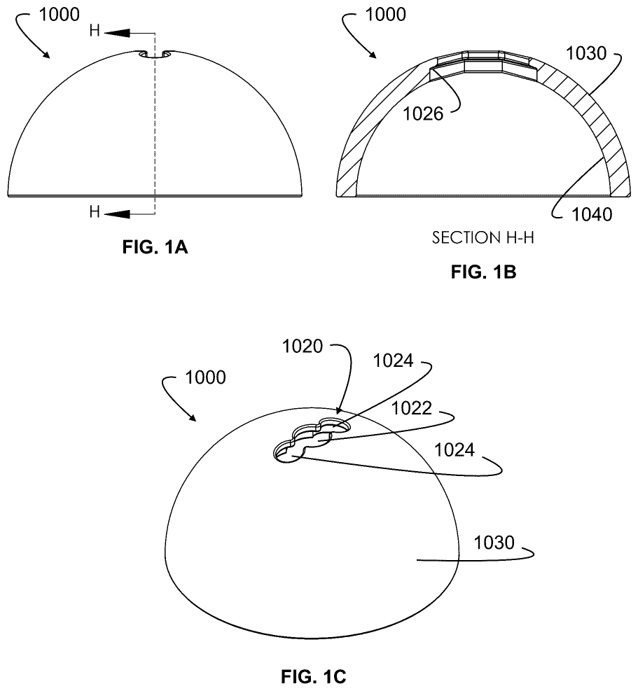

FIG. 1A is a side view of an acetabular cup 1000 according to an embodiment of the present disclosure. FIG. 1B is a front section view of the acetabular cup 1000 and FIG. 1C is a perspective view of the acetabular cup 1000. The acetabular cup 1000 may be configured to be received in a pelvis of a patient as part of a hip arthroplasty procedure. The acetabular cup 1000 may be configured to receive a natural femoral head as part of a ball and socket joint. Additionally, or alternatively, the acetabular cup 1000 may be configured to receive an anatomic prosthetic femoral head as part of a ball and socket joint.

An acetabular cup system may include an acetabular cup 1000, a central fastener 1100, and one or more locking fasteners 1150. The central fastener 1100 may be longer than the locking fasteners 1150. Alternatively, an acetabular cup system may include an acetabular cup 1000, a plurality of central fasteners 1100, and a plurality of locking fasteners 1150. Each of the plurality of central fasteners 1100 may be longer than the locking fasteners 1150.

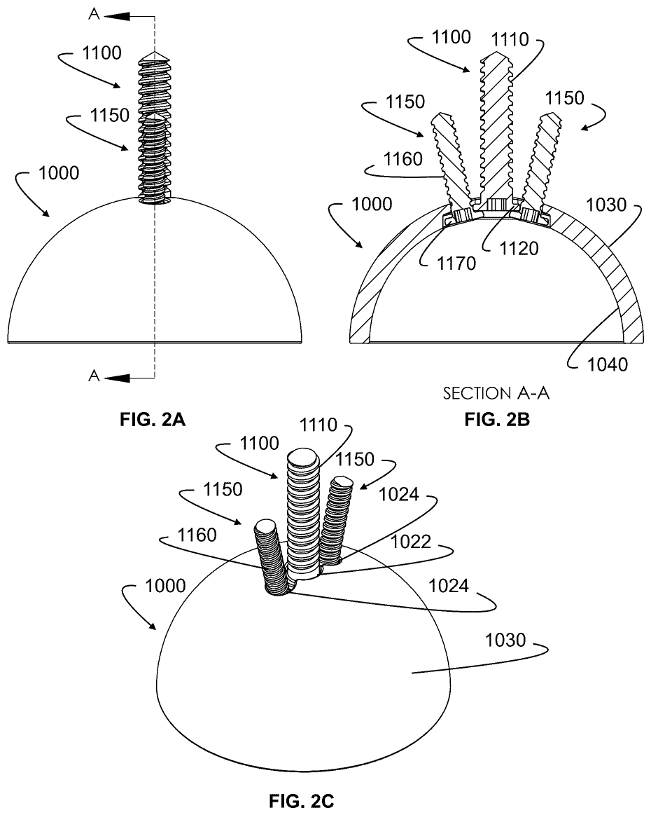

FIG. 2A is a side view of an acetabular cup 1000 a central fastener 1100, and two locking fasteners 1150 according to an embodiment of the present disclosure. FIG. 2B is a front section view of the acetabular cup 1000, the central fastener 1100, and the two locking fasteners 1150. FIG. 2C is a perspective view of the acetabular cup 1000, the central fastener 1100, and the two locking fasteners 1150. The acetabular cup 1000 may include a plurality of lobed fastener apertures 1020 configured to receive the central fastener 1100 and/or the locking fasteners 1150 within a single lobed fastener aperture 1020. At least one of the plurality of lobed fastener apertures 1020 may be configured to receive the central fastener 1100 and at least one of the locking fasteners 1150.

The central fastener 1100 may include a proximal end having a threaded portion 1110 and a distal end having a fastener head 1120. The locking fasteners 1150 may include a proximal end having a threaded portion 1160 and a distal end having a fastener head 1170. The plurality of lobed fastener apertures 1020 may be configured so that, with the threaded portion 1110 anchored in the pelvis 500, and the threaded portion 1160 anchored in the pelvis 500, the fastener head 1120 and the fastener head 1170 are adjacent, and the threaded portion 1110 and the threaded portion 1160 diverge within the pelvis 500.

Additionally, or alternatively, the plurality of lobed fastener apertures 1020 may be configured so that, with a threaded portion 1160 of a first of the locking fasteners 1150 anchored in the pelvis 500, and the threaded portion 1160 of a second of the locking fasteners 1150 anchored in the pelvis 500, the fastener head 1170 of the first of the locking fasteners 1150 and the fastener head 1170 of the second of the locking fasteners 1150 are adjacent, and the threaded portion 1160 of the first of the locking fasteners 1150 and the threaded portion 1160 of the second of the locking fasteners 1150 diverge within the pelvis 500.

The acetabular cup 1000 may include a plurality of lobed fastener apertures 1020. For clarity, an embodiment with a single lobed fastener aperture 1020 located at an apex of the acetabular cup 1000 is shown in FIGS. 2A-2C. In other embodiments, the acetabular cup 1000 may include two lobed fastener apertures 1020, three lobed fastener apertures 1020, four lobed fastener apertures 1020, five lobed fastener apertures 1020, six lobed fastener apertures 1020, or more than six lobed fastener apertures 1020.

For embodiments having two or more lobed fastener apertures 1020, the lobed fastener apertures 1020 may be spaced equally around a bone engaging surface 1030 of the acetabular cup 1000. Additionally, or alternatively, one instance of the lobed fastener apertures 1020 may be located at the apex of the acetabular cup 1000. Additionally, or alternatively, the orientation of the lobed fastener apertures 1020 may differ at each instance along the acetabular cup 1000.

In an embodiment, the central fastener 1100 may be configured as a bone screw including a threaded portion 1110 and a fastener head 1120 and may be insertable through the lobed fastener apertures 1020 and may be configured to be anchored in a pelvis 500 to secure the acetabular cup 1000 to the pelvis 500. The threaded portion 1110 may include a cancellous thread form configured to engage a portion of cancellous bone. In other embodiments, the central fastener 1100 may be configured as a rivet, a brad, a nail, a bolt, a post, a peg, a staple, an anchor, a line, a flexible member, a cable, a wire or another type of fastener known in the art may be used to provide fixation of the acetabular cup 1000 to a portion of bone.

In an embodiment, at least one of the locking fasteners 1150 may be configured as a bone screw including a threaded portion 1160 and a fastener head 1170 and may be insertable through the lobed fastener apertures 1020 and may be configured to be anchored in a pelvis 500 to secure the acetabular cup 1000 to the pelvis 500. The threaded portion 1160 may include a cancellous thread form configured to engage a portion of cancellous bone. In other embodiments, at least one of the locking fasteners 1150 may be configured as a rivet, a brad, a nail, a bolt, a post, a peg, a staple, an anchor, a line, a flexible member, a cable, a wire or another type of fastener known in the art may be used to provide fixation of the acetabular cup 1000 to a portion of bone.

At least one of the plurality of lobed fastener apertures 1020 may include a central aperture 1022 and at least one aperture lobe 1024. The central aperture 1022 may be configured to receive a central fastener 1100 and the at least one aperture lobe 1024 may be configured to receive at least one of the locking fasteners 1150. The at least one aperture lobe 1024 may intersect with the central aperture 1022. The central aperture 1022 may be larger than the aperture lobes 1024.

The acetabular cup 1000 may include an articulating surface 1040 configured to receive a natural femoral head as part of a ball and socket joint. Additionally, or alternatively, the articulating surface 1040 may be configured to receive an anatomic prosthetic femoral head as part of a ball and socket joint. The acetabular cup 1000 may further include a bone engaging surface 1030 configured to be received in a pelvis 500 of a patient.

The plurality of lobed fastener apertures 1020 may extend from a first opening at the articulating surface 1040 to a second opening at the bone engaging surface 1030. The first opening may include the central aperture 1022 and each of the one or more aperture lobes 1024. The second opening may also include the central aperture 1022 and each of the one or more aperture lobes 1024.

The first opening at the articulating surface 1040 may be larger than the second opening at the bone engaging surface 1030, thereby creating an aperture step 1026. The aperture step 1026 may be configured to engage a fastener head 1120 of the central fasteners 1100 and/or a fastener head 1170 of the locking fasteners 1150, thereby allowing the central fastener 1100 and/or the locking fasteners 1150 to secure the acetabular cup 1000 to the pelvis 500.

The central aperture 1022 and the fastener head 1120 of the central fastener 1100 may be configured so that the fastener head 1120 seats at a first depth within the lobed fastener apertures 1020. The aperture lobe 1024 and the fastener head 1170 of the locking fastener 1150 may be configured so that the fastener head 1170 seats at a second depth within the lobed fastener apertures 1020. The lobed fastener apertures 1020, the central fastener 1100, and the locking fastener 1150 may be configured so that the fastener head 1120 and the fastener head 1170 seat at different depths within the lobed fastener apertures 1020.

The plurality of lobed fastener apertures 1020 may further be configured so that when a central fastener 1100 is received in the central aperture 1022 and anchored in the pelvis 500, the fastener head 1120 does not protrude from the lobed fastener aperture 1020 past the articulating surface 1040. The plurality of lobed fastener apertures 1020 may further be configured so that when a locking fastener 1150 is received in an aperture lobe 1024 and anchored in the pelvis 500, the fastener head 1170 does not protrude from the lobed fastener aperture 1020 past the articulating surface 1040.

In an embodiment, the lobed fastener apertures 1020 may include one central aperture 1022 and two aperture lobes 1024. The central aperture 1022 may be configured to receive one central fastener 1100 and each of the two aperture lobes 1024 may be configured to receive a locking fastener 1150. The acetabular cup system may then include three bone fasteners and an acetabular cup 1000 having two aperture lobes 1024, wherein each of the two aperture lobes 1024 and the central aperture 1022 is configured to receive one of the three bone fasteners.

When the central fastener 1100 is received in the central aperture 1022 and anchored in the pelvis 500, and at least one locking fastener 1150 is received in at least one aperture lobe 1024 and anchored in the pelvis 500, the at least one of the locking fasteners 1150 may be configured to inhibit the central fastener 1100 from disengaging from the pelvis 500. Additionally, or alternatively, a fastener head 1170 of at least one of the locking fasteners 1150 may overlap a fastener head 1120 of the central fastener 1100, thereby inhibiting the central fastener 1100 from disengaging from the pelvis 500.

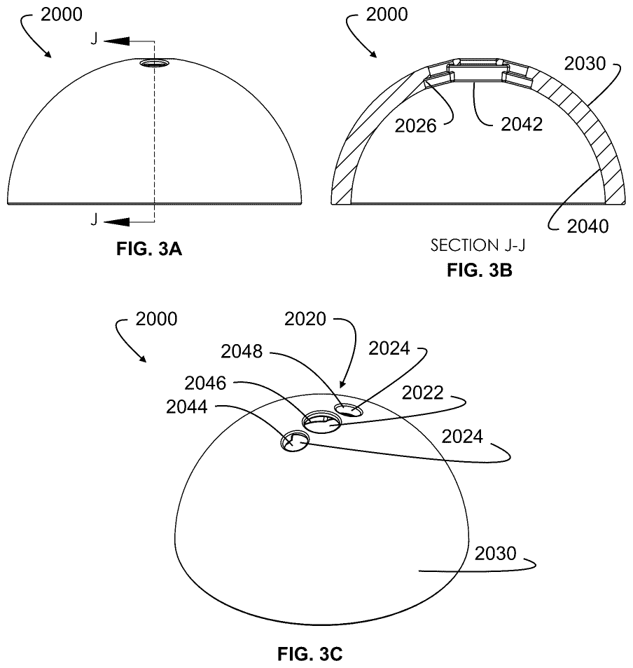

FIG. 3A is a side view of an acetabular cup 2000 according to an embodiment of the present disclosure. FIG. 3B is a front section view of the acetabular cup 2000 and FIG. 3C is a perspective view of the acetabular cup 2000. The acetabular cup 2000 may include similar function and features as the acetabular cup 1000. The acetabular cup 2000 may be configured to be received in a pelvis of a patient as part of a hip arthroplasty procedure. The acetabular cup 2000 may be configured to receive a natural femoral head as part of a ball and socket joint. Additionally, or alternatively, the acetabular cup 2000 may be configured to receive an anatomic prosthetic femoral head as part of a ball and socket joint.

An acetabular cup system may include an acetabular cup 2000, a central fastener 1100, and one or more locking fasteners 1150. The central fastener 1100 may be longer than the locking fasteners 1150. Alternatively, an acetabular cup system may include an acetabular cup 2000, a plurality of central fasteners 1100, and a plurality of locking fasteners 1150. Each of the plurality of central fasteners 1100 may be longer than the locking fasteners 1150.

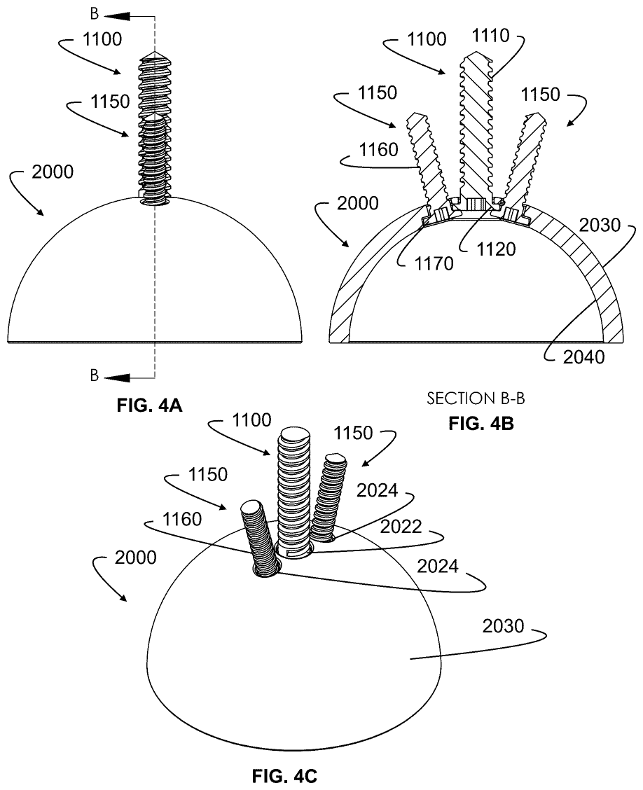

FIG. 4A is a side view of an acetabular cup 2000, a central fastener 1100, and two locking fasteners 1150 according to an embodiment of the present disclosure. FIG. 4B is a front section view of the acetabular cup 2000, the central fastener 1100, and the two locking fasteners 1150. FIG. 4C is a perspective view of the acetabular cup 2000, the central fastener 1100, and the two locking fasteners 1150. The acetabular cup 2000 may include a plurality of lobed fastener apertures 2020 configured to receive the central fastener 1100 and/or the locking fasteners 1150 within a single lobed fastener aperture 2020. At least one of the plurality of lobed fastener apertures 2020 may be configured to receive the central fastener 1100 and at least one of the locking fasteners 1150.

The central fastener 1100 may include a proximal end having a threaded portion 1110 and a distal end having a fastener head 1120. The locking fasteners 1150 may include a proximal end having a threaded portion 1160 and a distal end having a fastener head 1170. The plurality of lobed fastener apertures 2020 may be configured so that, with the threaded portion 1110 anchored in the pelvis 500, and the threaded portion 1160 anchored in the pelvis 500, the fastener head 1120 and the fastener head 1170 are adjacent, and the threaded portion 1110 and the threaded portion 1160 diverge within the pelvis 500.

Additionally, or alternatively, the plurality of lobed fastener apertures 2020 may be configured so that, with a threaded portion 1160 of a first of the locking fasteners 1150 anchored in the pelvis 500, and the threaded portion 1160 of a second of the locking fasteners 1150 anchored in the pelvis 500, the fastener head 1170 of the first of the locking fasteners 1150 and the fastener head 1170 of the second of the locking fasteners 1150 are adjacent, and the threaded portion 1160 of the first of the locking fasteners 1150 and the threaded portion 1160 of the second of the locking fasteners 1150 diverge within the pelvis 500.

The acetabular cup 2000 may include a plurality of lobed fastener apertures 2020. For clarity, an embodiment with a single lobed fastener aperture 2020 located at an apex of the acetabular cup 2000 is shown in FIGS. 4A-4C. In other embodiments, the acetabular cup 2000 may include two lobed fastener apertures 2020, three lobed fastener apertures 2020, four lobed fastener apertures 2020, five lobed fastener apertures 2020, six lobed fastener apertures 2020, or more than six lobed fastener apertures 2020.

For embodiments having two or more lobed fasteners apertures 2020, the lobed fastener apertures 2020 may be spaced equally around a bone engaging surface 2030 of the acetabular cup 2000. Additionally, or alternatively, one instance of the lobed fastener apertures 2020 may be located at the apex of the acetabular cup 2000. Additionally, or alternatively, the orientation of the lobed fastener apertures 2020 may differ at each instance along the acetabular cup 2000.

In an embodiment, the central fastener 1100 may be configured as a bone screw including a threaded portion 1110 and a fastener head 1120 and may be insertable through the lobed fastener apertures 2020 and may be configured to be anchored in a pelvis 500 to secure the acetabular cup 2000 to the pelvis 500. The threaded portion 1110 may include a cancellous thread form configured to engage a portion of cancellous bone. In other embodiments, the central fastener 1100 may be configured as a rivet, a brad, a nail, a bolt, a post, a peg, a staple, an anchor, a line, a flexible member, a cable, a wire or another type of fastener known in the art may be used to provide fixation of the acetabular cup 2000 to a portion of bone.

In an embodiment, at least one of the locking fasteners 1150 may be configured as a bone screw including a threaded portion 1160 and a fastener head 1170 and may be insertable through the lobed fastener apertures 2020 and may be configured to be anchored in a pelvis 500 to secure the acetabular cup 2000 to the pelvis 500. The threaded portion 1160 may include a cancellous thread form configured to engage a portion of cancellous bone. In other embodiments, at least one of the locking fasteners 1150 may be configured as a rivet, a brad, a nail, a bolt, a post, a peg, a staple, an anchor, a line, a flexible member, a cable, a wire or another type of fastener known in the art may be used to provide fixation of the acetabular cup 2000 to a portion of bone.

At least one of the plurality of lobed fastener apertures 2020 may include a central aperture 2022 and at least one aperture lobe 2024. The central aperture 2022 may be configured to receive a central fastener 1100 and the at least one aperture lobe 2024 may be configured to receive at least one of the locking fasteners 1150. The central aperture 2022 may be larger than the aperture lobes 2024.

The acetabular cup 2000 may include an articulating surface 2040 configured to receive a natural femoral head as part of a ball and socket joint. Additionally, or alternatively, the articulating surface 2040 may be configured to receive an anatomic prosthetic femoral head as part of a ball and socket joint. The acetabular cup 2000 may further include a bone engaging surface 2030 configured to be received in a pelvis 500 of a patient.

The plurality of lobed fastener apertures 2020 may extend from a first opening 2042 at the articulating surface 2040. The plurality of lobed fastener apertures 2020 may further include a second opening 2044, a third opening 2046, and a fourth opening 2048 at the bone engaging surface 2030. The first opening 2042 may include the central aperture 2022 and each of the one or more aperture lobes 2024. The second opening 2044 may include an aperture lobe 2024. The third opening 2046 may include the central aperture 2022. The fourth opening 2048 may include an aperture lobe 2024. The second opening 2044, the third opening 2046, and the fourth opening 2048 on the bone engaging surface 2030 may not intersect. The second opening 2044, the third opening 2046, and the fourth opening 2048 on the bone engaging surface 2030 may have a generally circular shape.

The first opening at the articulating surface 2040 may be larger than the second opening at the bone engaging surface 2030, thereby creating an aperture step 2026. The aperture step 2026 may be configured to engage a fastener head 1120 of the central fasteners 1100 and/or a fastener head 1170 of the locking fasteners 1150, thereby allowing the central fastener 1100 and/or the locking fasteners 1150 to secure the acetabular cup 2000 to the pelvis 500.

The central aperture 2022 and the fastener head 1120 of the central fastener 1100 may be configured so that the fastener head 1120 seats at a first depth within the lobed fastener apertures 2020. The aperture lobe 2024 and the fastener head 1170 of the locking fastener 1150 may be configured so that the fastener head 1170 seats at a second depth within the lobed fastener apertures 2020. The lobed fastener apertures 2020, the central fastener 1100, and the locking fastener 1150 may be configured so that the fastener head 1120 and the fastener head 1170 seat at different depths within the lobed fastener apertures 2020.

The plurality of lobed fastener apertures 2020 may further be configured so that when a central fastener 1100 is received in the central aperture 2022 and anchored in the pelvis 500, the fastener head 1120 does not protrude from the lobed fastener aperture 2020 past the articulating surface 2040. The plurality of lobed fastener apertures 2020 may further be configured so that when a locking fastener 1150 is received in an aperture lobe 2024 and anchored in the pelvis 500, the fastener head 1170 does not protrude from the lobed fastener aperture 2020 past the articulating surface 2040.

In an embodiment, the lobed fastener apertures 2020 may include one central aperture 2022 and two aperture lobes 2024. The central aperture 2022 may be configured to receive one central fastener 1100 and each of the two aperture lobes 2024 may be configured to receive a locking fastener 1150. The acetabular cup system may then include three bone fasteners and an acetabular cup 2000 having two aperture lobes 2024, wherein each of the two aperture lobes 2024 and the central aperture 2022 is configured to receive one of the three bone fasteners.

When the central fastener 1100 is received in the central aperture 2022 and anchored in the pelvis 500, and at least one locking fastener 1150 is received in at least one aperture lobe 2024 and anchored in the pelvis 500, the at least one of the locking fasteners 1150 may be configured to inhibit the central fastener 1100 from disengaging from the pelvis 500. Additionally, or alternatively, a fastener head 1170 of at least one of the locking fasteners 1150 may overlap a fastener head 1120 of the central fastener 1100, thereby inhibiting the central fastener 1100 from disengaging from the pelvis 500.

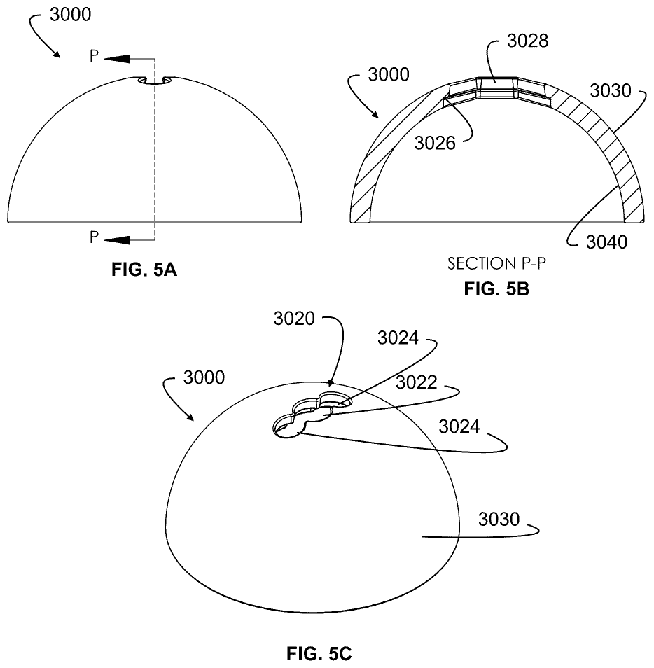

FIG. 5A is a side view of an acetabular cup 3000 according to an embodiment of the present disclosure. FIG. 5B is a front section view of the acetabular cup 3000 and FIG. 5C is a perspective view of the acetabular cup 3000. The acetabular cup 3000 may include similar function and features as the acetabular cup 1000. The acetabular cup 3000 may be configured to be received in a pelvis of a patient as part of a hip arthroplasty procedure. The acetabular cup 3000 may be configured to receive a natural femoral head as part of a ball and socket joint. Additionally, or alternatively, the acetabular cup 3000 may be configured to receive an anatomic prosthetic femoral head as part of a ball and socket joint.

An acetabular cup system may include an acetabular cup 3000, a central fastener 3100, and one or more locking fasteners 1150. The central fastener 3100 may be longer than the locking fasteners 1150. Alternatively, an acetabular cup system may include an acetabular cup 3000, a plurality of central fasteners 3100, and a plurality of locking fasteners 1150. Each of the plurality of central fasteners 3100 may be longer than the locking fasteners 1150.

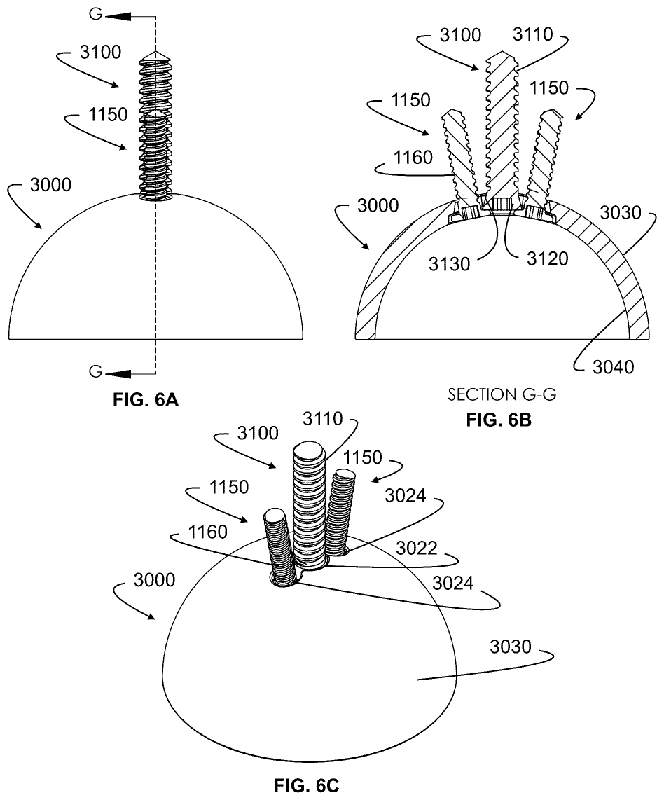

FIG. 6A is a side view of an acetabular cup 3000, a central fastener 3100, and two locking fasteners 1150 according to an embodiment of the present disclosure. FIG. 6B is a front section view of the acetabular cup 3000, the central fastener 3100, and the two locking fasteners 1150. FIG. 6C is a perspective view of the acetabular cup 3000, the central fastener 3100, and the two locking fasteners 1150. The acetabular cup 3000 may include a plurality of lobed fastener apertures 3020 configured to receive the central fastener 3100 and/or the locking fasteners 1150 within a single lobed fastener aperture 3020. At least one of the plurality of lobed fastener apertures 3020 may be configured to receive the central fastener 3100 and at least one of the locking fasteners 1150.

The central fastener 3100 may include a proximal end having a threaded portion 3110 and a distal end having a fastener head 3120. The locking fasteners 1150 may include a proximal end having a threaded portion 1160 and a distal end having a fastener head 1170. The plurality of lobed fastener apertures 3020 may be configured so that, with the threaded portion 3110 anchored in the pelvis 500, and the threaded portion 1160 anchored in the pelvis 500, the fastener head 3120 and the fastener head 1170 are adjacent, and the threaded portion 3110 and the threaded portion 1160 diverge within the pelvis 500.

Additionally, or alternatively, the plurality of lobed fastener apertures 3020 may be configured so that, with a threaded portion 1160 of a first of the locking fasteners 1150 anchored in the pelvis 500, and the threaded portion 1160 of a second of the locking fasteners 1150 anchored in the pelvis 500, the fastener head 1170 of the first of the locking fasteners 1150 and the fastener head 1170 of the second of the locking fasteners 1150 are adjacent, and the threaded portion 1160 of the first of the locking fasteners 1150 and the threaded portion 1160 of the second of the locking fasteners 1150 diverge within the pelvis 500.

The acetabular cup 3000 may include a plurality of lobed fastener apertures 3020. For clarity, an embodiment with a single lobed fastener aperture 3020 located at an apex of the acetabular cup 3000 is shown in FIGS. 6A-6C. In other embodiments, the acetabular cup 3000 may include two lobed fastener apertures 3020, three lobed fastener apertures 3020, four lobed fastener apertures 3020, five lobed fastener apertures 3020, six lobed fastener apertures 3020, or more than six lobed fastener apertures 3020.

For embodiments having two or more lobed fastener apertures 3020, the lobed fastener apertures 3020 may be spaced equally around a bone engaging surface 3030 of the acetabular cup 3000. Additionally, or alternatively, one instance of the lobed fastener apertures 3020 may be located at the apex of the acetabular cup 3000. Additionally, or alternatively, the orientation of the lobed fastener apertures 3020 may differ at each instance along the acetabular cup 3000.

In an embodiment, the central fastener 3100 may be configured as a bone screw including a threaded portion 3110 and a fastener head 3120 and may be insertable through the lobed fastener apertures 3020 and may be configured to be anchored in a pelvis 500 to secure the acetabular cup 3000 to the pelvis 500. The threaded portion 3110 may include a cancellous thread form configured to engage a portion of cancellous bone. In other embodiments, the central fastener 3100 may be configured as a rivet, a brad, a nail, a bolt, a post, a peg, a staple, an anchor, a line, a flexible member, a cable, a wire or another type of fastener known in the art may be used to provide fixation of the acetabular cup 3000 to a portion of bone.

In an embodiment, at least one of the locking fasteners 1150 may be configured as a bone screw including a threaded portion 1160 and a fastener head 1170 and may be insertable through the lobed fastener apertures 3020 and may be configured to be anchored in a pelvis 500 to secure the acetabular cup 3000 to the pelvis 500. The threaded portion 1160 may include a cancellous thread form configured to engage a portion of cancellous bone. In other embodiments, at least one of the locking fasteners 1150 may be configured as a rivet, a brad, a nail, a bolt, a post, a peg, a staple, an anchor, a line, a flexible member, a cable, a wire or another type of fastener known in the art may be used to provide fixation of the acetabular cup 3000 to a portion of bone.

At least one of the plurality of lobed fastener apertures 3020 may include a central aperture 3022 and at least one aperture lobe 3024. The central aperture 3022 may be configured to receive a central fastener 3100 and the at least one aperture lobe 3024 may be configured to receive at least one of the locking fasteners 1150. The at least one aperture lobe 3024 may intersect with the central aperture 3022. The central aperture 3022 may be larger than the aperture lobes 3024.

The acetabular cup 3000 may include an articulating surface 3040 configured to receive a natural femoral head as part of a ball and socket joint. Additionally, or alternatively, the articulating surface 3040 may be configured to receive an anatomic prosthetic femoral head as part of a ball and socket joint. The acetabular cup 3000 may further include a bone engaging surface 3030 configured to be received in a pelvis 500 of a patient.

The plurality of lobed fastener apertures 3020 may extend from a first opening at the articulating surface 3040. The plurality of lobed fastener apertures 3020 may further include a second opening at the bone engaging surface 3030. The first opening may include the central aperture 3022 and each of the one or more aperture lobes 3024. The second opening may also include the central aperture 3022 and each of the one or more aperture lobes 3024.

The first opening at the articulating surface 3040 may be larger than the second opening at the bone engaging surface 3030, thereby creating an aperture step 3026. The aperture step 3026 may be configured to engage a fastener head 3120 of the central fasteners 3100 and/or a fastener head 1170 of the locking fasteners 1150, thereby allowing the central fastener 3100 and/or the locking fasteners 1150 to secure the acetabular cup 3000 to the pelvis 500.

Additionally, or alternatively, the central aperture 3022 may include a central aperture threaded portion 3028. The fastener head 3120 of the central fastener 3100 may include a proximal threaded portion 3130. The proximal threaded portion 3130 may correspond to the central aperture threaded portion 3028.

The central aperture threaded portion 3028 may be configured as a tapered thread whereby the tapered thread is larger at the articulating surface 3040 than at the bone engaging surface 3030. The proximal threaded portion 3130 may include a tapered thread that may be complementary to the tapered thread of the central aperture threaded portion 3028 thereby allowing the central fastener 3100 to threadably engage the acetabular cup 3000 so that the fastener head 3120 is captive within the central aperture 3022 thereby allowing the central fastener 3100 to secure the acetabular cup 3000 to the pelvis 500.

The central aperture threaded portion 3028 may be configured to receive a central fastener 3100 having a proximal threaded portion 3130 and/or a central fastener 1100 without a proximal threaded portion.

The central aperture 3022 and the fastener head 3120 of the central fastener 3100 may be configured so that the fastener head 3120 seats at a first depth within the lobed fastener apertures 3020. The aperture lobe 3024 and the fastener head 1170 of the locking fastener 1150 may be configured so that the fastener head 1170 seats at a second depth within the lobed fastener apertures 3020. The lobed fastener apertures 3020, the central fastener 3100, and the locking fastener 1150 may be shaped and configured such that the fastener head 3120 and the fastener head 1170 seat at different depths within the lobed fastener apertures 3020.

The plurality of lobed fastener apertures 3020 may further be configured so that when a central fastener 3100 is received in the central aperture 3022 and anchored in the pelvis 500, the fastener head 3120 does not protrude from the lobed fastener aperture 3020 past the articulating surface 3040. The plurality of lobed fastener apertures 3020 may further be configured so that when a locking fastener 1150 is received in an aperture lobe 3024 and anchored in the pelvis 500, the fastener head 1170 does not protrude from the lobed fastener aperture 3020 past the articulating surface 3040.

In an embodiment, the lobed fastener apertures 3020 may include one central aperture 3022 and two aperture lobes 3024. The central aperture 3022 may be configured to receive one central fastener 3100 and each of the two aperture lobes 3024 may be configured to receive a locking fastener 1150. The acetabular cup system may then include three bone fasteners and an acetabular cup 3000 having two aperture lobes 3024, wherein each of the two aperture lobes 3024 and the central aperture 3022 is configured to receive one of the three bone fasteners.

When the central fastener 3100 is received in the central aperture 3022 and anchored in the pelvis 500, and at least one locking fastener 1150 is received in at least one aperture lobe 3024 and anchored in the pelvis 500, the at least one of the locking fasteners 1150 may be configured to inhibit the central fastener 3100 from disengaging from the pelvis 500. Additionally, or alternatively, a fastener head 1170 of at least one of the locking fasteners 1150 may overlap a fastener head 3120 of the central fastener 3100, thereby inhibiting the central fastener 3100 from disengaging from the pelvis 500.

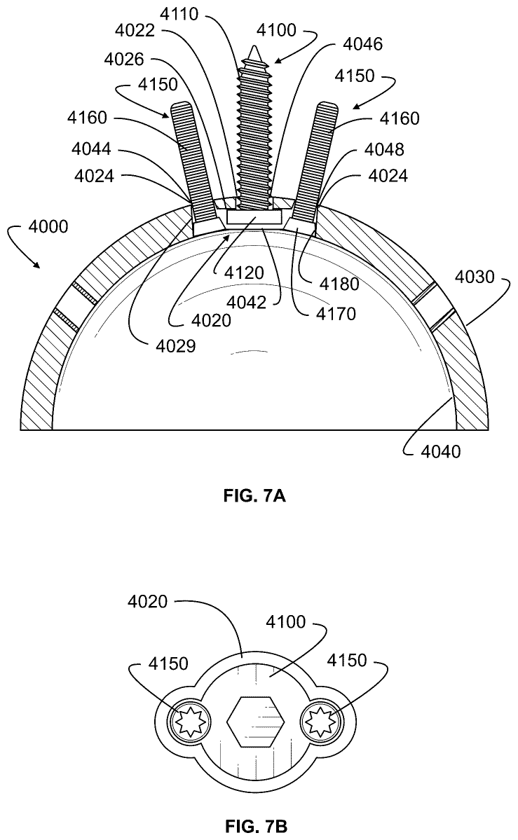

FIG. 7A is a front section view of an acetabular cup 4000, a central fastener 4100, and two locking fasteners 4150 according to an embodiment of the present disclosure. FIG. 7B is a bottom detail view of the central fastener 4100, and the two locking fasteners 4150. The acetabular cup 4000 may include similar function and features as the acetabular cup 1000. The acetabular cup 4000 may be configured to be received in a pelvis of a patient as part of a hip arthroplasty procedure. The acetabular cup 4000 may be configured to receive a natural femoral head as part of a ball and socket joint. Additionally, or alternatively, the acetabular cup 4000 may be configured to receive an anatomic prosthetic femoral head as part of a ball and socket joint.

An acetabular cup system may include an acetabular cup 4000, a central fastener 4100, and one or more locking fasteners 4150. The central fastener 4100 may be longer than the locking fasteners 4150. Alternatively, an acetabular cup system may include an acetabular cup 4000, a plurality of central fasteners 4100, and a plurality of locking fasteners 4150. Each of the plurality of central fasteners 4100 may be longer than the locking fasteners 4150.

The acetabular cup 4000 may include a plurality of lobed fastener apertures 4020 configured to receive the central fastener 4100 and/or the locking fasteners 4150 within a single lobed fastener aperture 4020. At least one of the plurality of lobed fastener apertures 4020 may be configured to receive the central fastener 4100 and at least one of the locking fasteners 4150.

The central fastener 4100 may include a proximal end having a threaded portion 4110 and a distal end having a fastener head 4120. The locking fasteners 4150 may include a proximal end having a threaded portion 4160 and a distal end having a fastener head 4170. The plurality of lobed fastener apertures 4020 may be configured so that, with the threaded portion 4110 anchored in the pelvis 500, and the threaded portion 4160 anchored in the pelvis 500, the fastener head 4120 and the fastener head 4170 are adjacent, and the threaded portion 4110 and the threaded portion 4160 diverge within the pelvis 500.

Additionally, or alternatively, the plurality of lobed fastener apertures 4020 may be configured so that, with a threaded portion 4160 of a first of the locking fasteners 4150 anchored in the pelvis 500, and the threaded portion 4160 of a second of the locking fasteners 4150 anchored in the pelvis 500, the fastener head 4170 of the first of the locking fasteners 4150 and the fastener head 4170 of the second of the locking fasteners 4150 are adjacent, and the threaded portion 4160 of the first of the locking fasteners 4150 and the threaded portion 4160 of the second of the locking fasteners 4150 diverge within the pelvis 500.

The acetabular cup 4000 may include a plurality of lobed fastener apertures 4020. For clarity, an embodiment with a single lobed fastener aperture 4020 located at an apex of the acetabular cup 4000 is shown in FIG. 7A. In other embodiments, the acetabular cup 4000 may include two lobed fastener apertures 4020, three lobed fastener apertures 4020, four lobed fastener apertures 4020, five lobed fastener apertures 4020, six lobed fastener apertures 4020, or more than six lobed fastener apertures 4020.

For embodiments having two or more lobed fastener apertures 4020, the lobed fastener apertures 4020 may be spaced equally around a bone engaging surface 4030 of the acetabular cup 4000. Additionally, or alternatively, one instance of the lobed fastener apertures 4020 may be located at the apex of the acetabular cup 4000. Additionally, or alternatively, the orientation of the lobed fastener apertures 4020 may differ at each instance along the acetabular cup 4000.

In an embodiment, the central fastener 4100 may be configured as a bone screw including a threaded portion 4110 and a fastener head 4120 and may be insertable through the lobed fastener apertures 4020 and may be configured to be anchored in a pelvis 500 to secure the acetabular cup 4000 to the pelvis 500. The threaded portion 4110 may include a cancellous thread form configured to engage a portion of cancellous bone. In other embodiments, the central fastener 4100 may be configured as a rivet, a brad, a nail, a bolt, a post, a peg, a staple, an anchor, a line, a flexible member, a cable, a wire or another type of fastener known in the art may be used to provide fixation of the acetabular cup 4000 to a portion of bone.

In an embodiment, at least one of the locking fasteners 4150 may be configured as a bone screw including a threaded portion 4160 and a fastener head 4170 and may be insertable through the lobed fastener apertures 4020 and may be configured to be anchored in a pelvis 500 to secure the acetabular cup 4000 to the pelvis 500. The threaded portion 4160 may include a cancellous thread form configured to engage a portion of cancellous bone. In other embodiments, at least one of the locking fasteners 4150 may be configured as a rivet, a brad, a nail, a bolt, a post, a peg, a staple, an anchor, a line, a flexible member, a cable, a wire or another type of fastener known in the art may be used to provide fixation of the acetabular cup 4000 to a portion of bone.

At least one of the plurality of lobed fastener apertures 4020 may include a central aperture 4022 and at least one aperture lobe 4024. The central aperture 4022 may be configured to receive a central fastener 4100 and the at least one aperture lobe 4024 may be configured to receive at least one of the locking fasteners 4150. The at least one aperture lobe 4024 may intersect with the central aperture 4022. The central aperture 4022 may be larger than the aperture lobes 4024.

The acetabular cup 4000 may include an articulating surface 4040 configured to receive a natural femoral head as part of a ball and socket joint. Additionally, or alternatively, the articulating surface 4040 may be configured to receive an anatomic prosthetic femoral head as part of a ball and socket joint. The acetabular cup 4000 may further include a bone engaging surface 4030 configured to be received in a pelvis 500 of a patient.

The plurality of lobed fastener apertures 4020 may extend from a first opening 4042 at the articulating surface 2040. The plurality of lobed fastener apertures 4020 may further include a second opening 4044, a third opening 4046, and a fourth opening 4048 at the bone engaging surface 4030. The first opening 4042 may include the central aperture 4022 and each of the one or more aperture lobes 4024. The second opening 4044 may include an aperture lobe 4024. The third opening 4046 may include the central aperture 4022. The fourth opening 4048 may include an aperture lobe 4024. The second opening 4044, the third opening 4046, and the fourth opening 4048 on the bone engaging surface 4030 may not intersect.

The first opening at the articulating surface 4040 may be larger than the second opening at the bone engaging surface 4030, thereby creating an aperture step 4026. The aperture step 4026 may be configured to engage a fastener head 4120 of the central fasteners 4100 and/or a fastener head 4170 of the locking fasteners 4150, thereby allowing the central fastener 4100 and/or the locking fasteners 4150 to secure the acetabular cup 4000 to the pelvis 500.

Additionally, or alternatively, the aperture lobe 4024 may include an aperture lobe threaded portion 4029. The fastener head 4170 of the locking fastener 4150 may include a proximal threaded portion 4180. The proximal threaded portion 4180 may correspond to the aperture lobe threaded portion 4029.

The aperture lobe threaded portion 4029 may be configured to receive a locking fastener 4150 having a proximal threaded portion 4180 and/or a locking fastener 1150 without a proximal threaded portion.

The aperture lobe threaded portion 4029 may be configured as a tapered thread whereby the tapered thread is larger at the articulating surface 4040 than at the bone engaging surface 4030. The proximal threaded portion 4180 may include a tapered thread that may be complementary to the tapered thread of the aperture lobe threaded portion 4029 thereby allowing the locking fastener 4150 to threadably engage the acetabular cup 4000 so that the fastener head 4170 is captive within the aperture lobe 4024 thereby allowing the locking fastener 4150 to secure the acetabular cup 4000 to the pelvis 500.

The central aperture 4022 and the fastener head 4120 of the central fastener 4100 may be configured so that the fastener head 4120 seats at a first depth within the lobed fastener apertures 4020. The aperture lobe 4024 and the fastener head 4170 of the locking fastener 4150 may be configured so that the fastener head 4170 seats at a second depth within the lobed fastener apertures 4020. The lobed fastener apertures 4020, the central fastener 4100, and the locking fastener 4150 may be shaped and configured such that the fastener head 4120 and the fastener head 4170 seat at different depths within the lobed fastener apertures 4020.

The plurality of lobed fastener apertures 4020 may further be configured so that when a central fastener 4100 is received in the central aperture 4022 and anchored in the pelvis 500, the fastener head 4120 does not protrude from the lobed fastener aperture 4020 past the articulating surface 4040. The plurality of lobed fastener apertures 4020 may further be configured so that when a locking fastener 4150 is received in an aperture lobe 4024 and anchored in the pelvis 500, the fastener head 4170 does not protrude from the lobed fastener aperture 4020 past the articulating surface 4040.

In an embodiment, the lobed fastener apertures 4020 may include one central aperture 4022 and two aperture lobes 4024. The central aperture 4022 may be configured to receive one central fastener 4100 and each of the two aperture lobes 4024 may be configured to receive a locking fastener 4150. The acetabular cup system may then include three bone fasteners and an acetabular cup 4000 having two aperture lobes 4024, wherein each of the two aperture lobes 4024 and the central aperture 4022 is configured to receive one of the three bone fasteners.

When the central fastener 4100 is received in the central aperture 4022 and anchored in the pelvis 500, and at least one locking fastener 4150 is received in at least one aperture lobe 4024 and anchored in the pelvis 500, the at least one of the locking fasteners 4150 may be configured to inhibit the central fastener 4100 from disengaging from the pelvis 500. Additionally, or alternatively, a fastener head 4170 of at least one of the locking fasteners 4150 may overlap a fastener head 4120 of the central fastener 4100, thereby inhibiting the central fastener 4100 from disengaging from the pelvis 500.

Additionally, or alternatively, the plurality of lobed fastener apertures 4020, the central fastener 4100, and the locking fasteners 4150 may be configured so that, with a threaded portion 4160 of a first of the locking fasteners 4150 anchored in the pelvis 500, the threaded portion 4160 of a second of the locking fasteners 4150 anchored in the pelvis 500, and the threaded portion 4110 of the central fastener 4100 anchored in the pelvis, the fastener head 1170 of the first of the locking fasteners 1150, the fastener head 1170 of the second of the locking fasteners 1150, and the fastener head 4120 of the central fastener 4100 do not touch each other.

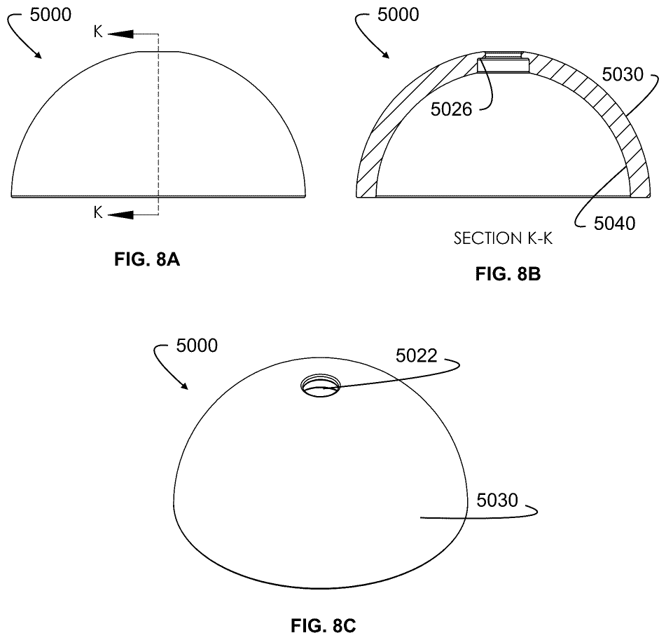

FIG. 8A is a side view of an acetabular cup 5000 according to an embodiment of the present disclosure. FIG. 8B is a front section view of the acetabular cup 5000 and FIG. 8C is a perspective view of the acetabular cup 5000. The acetabular cup 5000 may include similar function and features as the acetabular cup 5000. The acetabular cup 5000 may be configured to be received in a pelvis of a patient as part of a hip arthroplasty procedure. The acetabular cup 5000 may be configured to receive a natural femoral head as part of a ball and socket joint. Additionally, or alternatively, the acetabular cup 5000 may be configured to receive an anatomic prosthetic femoral head as part of a ball and socket joint.

An acetabular cup system may include an acetabular cup 5000 and a central fastener 1100. Alternatively, an acetabular cup system may include an acetabular cup 5000 and a plurality of central fasteners 1100.

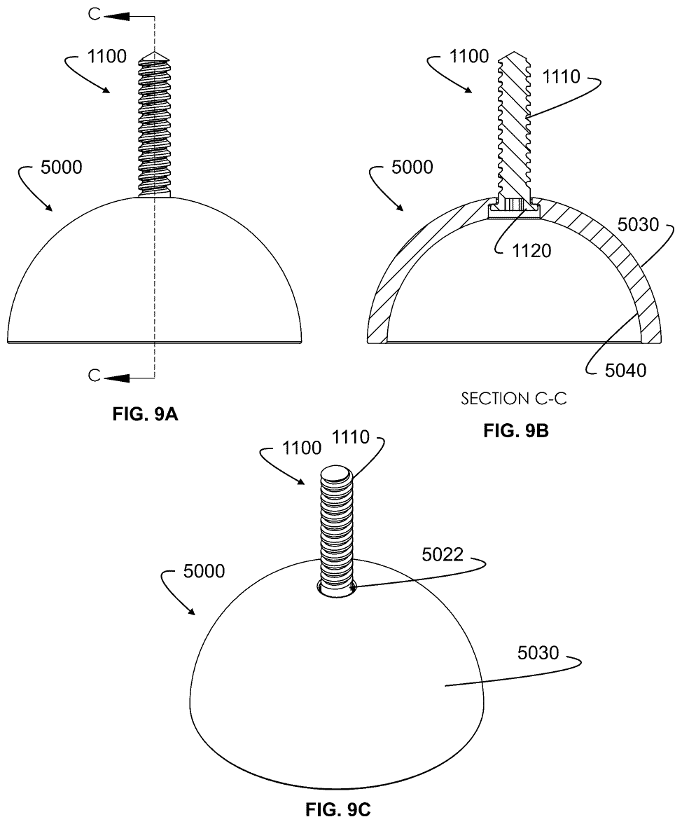

FIG. 9A is a side view of an acetabular cup 5000 and a central fastener 1100 according to an embodiment of the present disclosure. FIG. 9B is a front section view of the acetabular cup 5000 and the central fastener 1100. FIG. 9C is a perspective view of the acetabular cup 5000 and the central fastener 1100. The acetabular cup 5000 may include a plurality of central apertures 5022 configured to receive the central fastener 1100.

The central fastener 1100 may include a proximal end having a threaded portion 1110 and a distal end having a fastener head 1120.

The acetabular cup 5000 may include a plurality of central apertures 5022. For clarity, an embodiment with a single central aperture 5022 located at an apex of the acetabular cup 5000 is shown in FIGS. 9A-9C. In other embodiments, the acetabular cup 5000 may include two central apertures 5022, three central apertures 5022, four central apertures 5022, five central apertures 5022, six central apertures 5022, or more than six central apertures 5022.

For embodiments having two or more central apertures 5022, the central apertures 5022 may be spaced equally around a bone engaging surface 5030 of the acetabular cup 5000. Additionally, or alternatively, one instance of the central apertures 5022 may be located at the apex of the acetabular cup 5000.

In an embodiment, the central fastener 1100 may be configured as a bone screw including a threaded portion 1110 and a fastener head 1120 and may be insertable through the central aperture 5022 and may be configured to be anchored in a pelvis 500 to secure the acetabular cup 5000 to the pelvis 500. The threaded portion 1110 may include a cancellous thread form configured to engage a portion of cancellous bone. In other embodiments, the central fastener 1100 may be configured as a rivet, a brad, a nail, a bolt, a post, a peg, a staple, an anchor, a line, a flexible member, a cable, a wire or another type of fastener known in the art may be used to provide fixation of the acetabular cup 5000 to a portion of bone.

The acetabular cup 5000 may include an articulating surface 5040 configured to receive a natural femoral head as part of a ball and socket joint. Additionally, or alternatively, the articulating surface 5040 may be configured to receive an anatomic prosthetic femoral head as part of a ball and socket joint. The acetabular cup 5000 may further include a bone engaging surface 5030 configured to be received in a pelvis 500 of a patient.

The central aperture 5022 may extend from a first opening at the articulating surface 5040. The central aperture 5022 may further include a second opening at the bone engaging surface 5030. The first opening at the articulating surface 5040 may be larger than the second opening at the bone engaging surface 5030, thereby creating an aperture step 5026. The aperture step 5026 may be configured to engage a fastener head 1120 of the central fasteners 1100, thereby allowing the central fastener 1100 to secure the acetabular cup 5000 to the pelvis 500.

The central aperture 5022 may further be configured so that when a central fastener 1100 is received in the central aperture 5022 and anchored in the pelvis 500, the fastener head 1120 does not protrude from the central aperture 5022 past the articulating surface 5040.

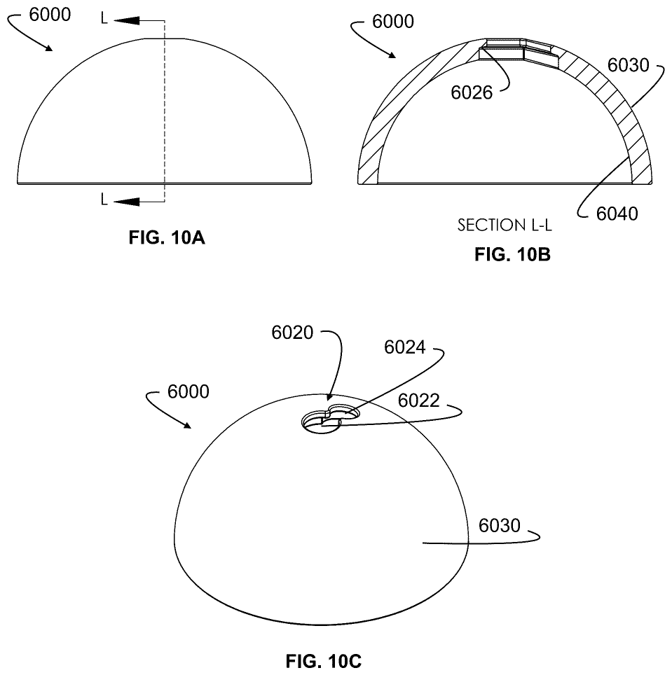

FIG. 10A is a side view of an acetabular cup 6000 according to an embodiment of the present disclosure. FIG. 10B is a front section view of the acetabular cup 6000 and FIG. 10C is a perspective view of the acetabular cup 6000. The acetabular cup 6000 may include similar function and features as the acetabular cup 1000. The acetabular cup 6000 may be configured to be received in a pelvis of a patient as part of a hip arthroplasty procedure. The acetabular cup 6000 may be configured to receive a natural femoral head as part of a ball and socket joint. Additionally, or alternatively, the acetabular cup 6000 may be configured to receive an anatomic prosthetic femoral head as part of a ball and socket joint.

An acetabular cup system may include an acetabular cup 6000, a central fastener 1100 and a locking fastener 1150. The central fastener 1100 may be longer than the locking fastener 1150. Alternatively, an acetabular cup system may include an acetabular cup 6000, a plurality of central fasteners 1100, and a plurality of locking fasteners 1150. Each of the plurality of central fasteners 1100 may be longer than the locking fasteners 1150.

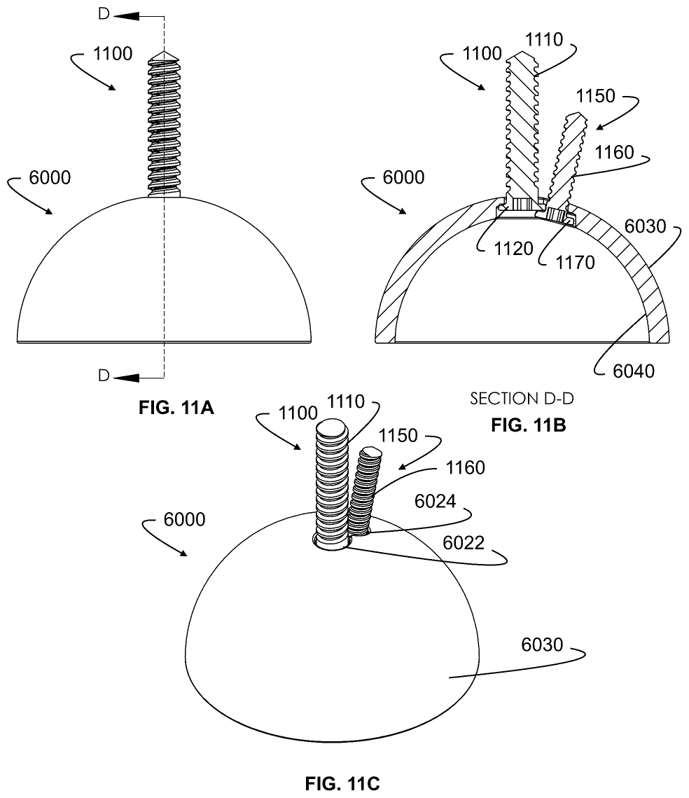

FIG. 11A is a side view of an acetabular cup 6000, a central fastener 1100, and a locking fastener 1150 according to an embodiment of the present disclosure. FIG. 11B is a front section view of the acetabular cup 6000, the central fastener 1100, and the locking fastener 1150. FIG. 11C is a perspective view of the acetabular cup 3000, the central fastener 3100, and the locking fastener 1150. The acetabular cup 6000 may include a plurality of lobed fastener apertures 6020 configured to receive the central fastener 1100 and/or the locking fastener 1150 within a single lobed fastener aperture 6020. At least one of the plurality of lobed fastener apertures 6020 may be configured to receive the central fastener 1100 and the locking fastener 1150.

The central fastener 1100 may include a proximal end having a threaded portion 1110 and a distal end having a fastener head 1120. The locking fastener 1150 may include a proximal end having a threaded portion 1160 and a distal end having a fastener head 1170. The plurality of lobed fastener apertures 6020 may be configured so that, with the threaded portion 1110 anchored in the pelvis 500, and the threaded portion 1160 anchored in the pelvis 500, the fastener head 1120 and the fastener head 1170 are adjacent, and the threaded portion 1110 and the threaded portion 1160 diverge within the pelvis 500.

The acetabular cup 6000 may include a plurality of lobed fastener apertures 6020. For clarity, an embodiment with a single lobed fastener aperture 6020 located at an apex of the acetabular cup 6000 is shown in FIGS. 11A-11C. In other embodiments, the acetabular cup 6000 may include two lobed fastener apertures 6020, three lobed fastener apertures 6020, four lobed fastener apertures 6020, five lobed fastener apertures 6020, six lobed fastener apertures 6020, or more than six lobed fastener apertures 6020.

For embodiments having two or more lobed fastener apertures 6020, the lobed fastener apertures 6020 may be spaced equally around a bone engaging surface 6030 of the acetabular cup 6000. Additionally, or alternatively, one instance of the lobed fastener apertures 6020 may be located at the apex of the acetabular cup 6000. Additionally, or alternatively, the orientation of the lobed fastener apertures 6020 may differ at each instance along the acetabular cup 6000.

In an embodiment, the central fastener 1100 may be configured as a bone screw including a threaded portion 1110 and a fastener head 1120 and may be insertable through the lobed fastener apertures 6020 and may be configured to be anchored in a pelvis 500 to secure the acetabular cup 6000 to the pelvis 500. The threaded portion 1110 may include a cancellous thread form configured to engage a portion of cancellous bone. In other embodiments, the central fastener 1100 may be configured as a rivet, a brad, a nail, a bolt, a post, a peg, a staple, an anchor, a line, a flexible member, a cable, a wire or another type of fastener known in the art may be used to provide fixation of the acetabular cup 6000 to a portion of bone.

In an embodiment, the locking fastener 1150 may be configured as a bone screw including a threaded portion 1160 and a fastener head 1170 and may be insertable through the lobed fastener apertures 3020 and may be configured to be anchored in a pelvis 500 to secure the acetabular cup 6000 to the pelvis 500. The threaded portion 1160 may include a cancellous thread form configured to engage a portion of cancellous bone. In other embodiments, the locking fastener 1150 may be configured as a rivet, a brad, a nail, a bolt, a post, a peg, a staple, an anchor, a line, a flexible member, a cable, a wire or another type of fastener known in the art may be used to provide fixation of the acetabular cup 6000 to a portion of bone.

At least one of the plurality of lobed fastener apertures 6020 may include a central aperture 6022 and an aperture lobe 6024. The central aperture 6022 may be configured to receive a central fastener 1100 and the aperture lobe 6024 may be configured to receive the locking fastener 1150. The aperture lobe 6024 may intersect with the central aperture 6022. The central aperture 6022 may be larger than the aperture lobe 6024.

The acetabular cup 6000 may include an articulating surface 6040 configured to receive a natural femoral head as part of a ball and socket joint. Additionally, or alternatively, the articulating surface 6040 may be configured to receive an anatomic prosthetic femoral head as part of a ball and socket joint. The acetabular cup 6000 may further include a bone engaging surface 6030 configured to be received in a pelvis 500 of a patient.

The plurality of lobed fastener apertures 6020 may extend from a first opening at the articulating surface 6040. The plurality of lobed fastener apertures 6020 may further include a second opening at the bone engaging surface 6030. The first opening may include the central aperture 6022 and the aperture lobe 6024. The second opening may also include the central aperture 6022 and the aperture lobe 6024.

The first opening at the articulating surface 6040 may be larger than the second opening at the bone engaging surface 6030, thereby creating an aperture step 6026. The aperture step 6026 may be configured to engage a fastener head 1120 of the central fasteners 1100 and/or a fastener head 1170 of the locking fastener 1150, thereby allowing the central fastener 1100 and/or the locking fastener 1150 to secure the acetabular cup 6000 to the pelvis 500.

The central aperture 6022 and the fastener head 1120 of the central fastener 1100 may be configured so that the fastener head 1120 seats at a first depth within the lobed fastener apertures 6020. The aperture lobe 6024 and the fastener head 1170 of the locking fastener 1150 may be configured so that the fastener head 1170 seats at a second dept within the lobed fastener apertures 6020. The lobed fastener apertures 6020, the central fastener 1100, and the locking fastener 1150 may be shaped and configured such that the fastener head 1120 and the fastener head 1170 seat at different depths within the lobed fastener apertures 6020.

The plurality of lobed fastener apertures 6020 may further be configured so that when a central fastener 1100 is received in the central aperture 6022 and anchored in the pelvis 500, the fastener head 1120 does not protrude from the lobed fastener aperture 6020 past the articulating surface 6040. The plurality of lobed fastener apertures 6020 may further be configured so that when the locking fastener 1150 is received in the aperture lobe 6024 and anchored in the pelvis 500, the fastener head 1170 does not protrude from the lobed fastener aperture 6020 past the articulating surface 6040.

When the central fastener 1100 is received in the central aperture 6022 and anchored in the pelvis 500, and the locking fastener 1150 is received in the aperture lobe 6024 and anchored in the pelvis 500, the locking fastener 1150 may be configured to inhibit the central fastener 1100 from disengaging from the pelvis 500. Additionally, or alternatively, a fastener head 1170 of the locking fastener 1150 may overlap a fastener head 1120 of the central fastener 1100, thereby inhibiting the central fastener 1100 from disengaging from the pelvis 500.

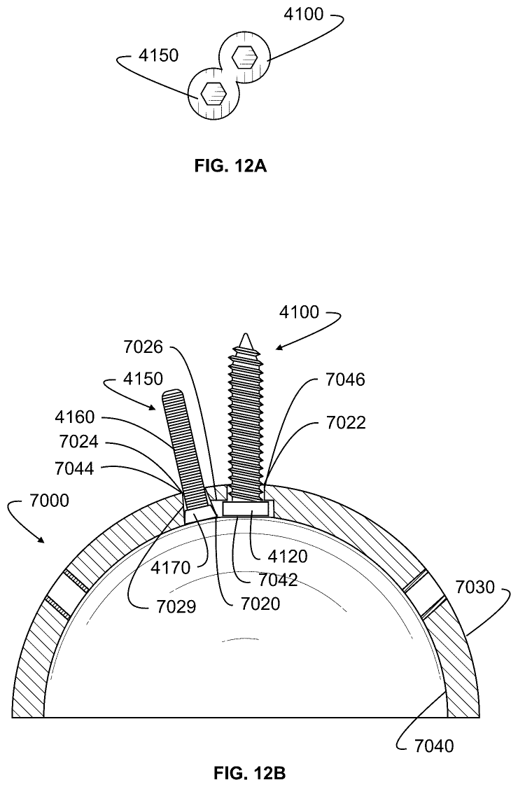

FIG. 12A is a bottom detail view of a central fastener 4100 and a locking fastener 4150 according to an embodiment of the present disclosure. FIG. 12B is a front section view of an acetabular cup 7000 and the central fastener 4100 and the locking fastener 4150. The acetabular cup 7000 may include similar function and features as the acetabular cup 1000. The acetabular cup 7000 may be configured to be received in a pelvis of a patient as part of a hip arthroplasty procedure. The acetabular cup 7000 may be configured to receive a natural femoral head as part of a ball and socket joint. Additionally, or alternatively, the acetabular cup 7000 may be configured to receive an anatomic prosthetic femoral head as part of a ball and socket joint.

An acetabular cup system may include an acetabular cup 7000, a central fastener 4100, and a locking fasteners 4150. The central fastener 4100 may be longer than the locking fastener 4150. Alternatively, an acetabular cup system may include an acetabular cup 7000, a plurality of central fasteners 4100, and a plurality of locking fasteners 4150. Each of the plurality of central fasteners 4100 may be longer than the locking fasteners 4150.

The acetabular cup 7000 may include a plurality of lobed fastener apertures 7020 configured to receive the central fastener 4100 and/or the locking fastener 4150 within a single lobed fastener aperture 7020. At least one of the plurality of lobed fastener apertures 7020 may be configured to receive the central fastener 4100 and the locking fastener 4150.

The central fastener 4100 may include a proximal end having a threaded portion 4110 and a distal end having a fastener head 4120. The locking fastener 4150 may include a proximal end having a threaded portion 4160 and a distal end having a fastener head 4170. The plurality of lobed fastener apertures 7020 may be configured so that, with the threaded portion 4110 anchored in the pelvis 500, and the threaded portion 4160 anchored in the pelvis 500, the fastener head 4120 and the fastener head 4170 are adjacent, and the threaded portion 4110 and the threaded portion 4160 diverge within the pelvis 500.

The acetabular cup 7000 may include a plurality of lobed fastener apertures 7020. For clarity, an embodiment with a single lobed fastener aperture 7020 located at an apex of the acetabular cup 7000 is shown in FIG. 12B. In other embodiments, the acetabular cup 7000 may include two lobed fastener apertures 7020, three lobed fastener apertures 7020, four lobed fastener apertures 7020, five lobed fastener apertures 7020, six lobed fastener apertures 7020, or more than six lobed fastener apertures 7020.

For embodiments having two or more lobed fastener apertures 7020, the lobed fastener apertures 7020 may be spaced equally around a bone engaging surface 7030 of the acetabular cup 7000. Additionally, or alternatively, one instance of the lobed fastener apertures 7020 may be located at the apex of the acetabular cup 7000. Additionally, or alternatively, the orientation of the lobed fastener apertures 7020 may differ at each instance along the acetabular cup 7000.

In an embodiment, the central fastener 4100 may be configured as a bone screw including a threaded portion 4110 and a fastener head 4120 and may be insertable through the lobed fastener apertures 7020 and may be configured to be anchored in a pelvis 500 to secure the acetabular cup 7000 to the pelvis 500. The threaded portion 4110 may include a cancellous thread form configured to engage a portion of cancellous bone. In other embodiments, the central fastener 4100 may be configured as a rivet, a brad, a nail, a bolt, a post, a peg, a staple, an anchor, a line, a flexible member, a cable, a wire or another type of fastener known in the art may be used to provide fixation of the acetabular cup 7000 to a portion of bone.

In an embodiment, the locking fastener 4150 may be configured as a bone screw including a threaded portion 4160 and a fastener head 4170 and may be insertable through the lobed fastener apertures 7020 and may be configured to be anchored in a pelvis 500 to secure the acetabular cup 4000 to the pelvis 500. The threaded portion 4160 may include a cancellous thread form configured to engage a portion of cancellous bone. In other embodiments, at least one of the locking fasteners 4150 may be configured as a rivet, a brad, a nail, a bolt, a post, a peg, a staple, an anchor, a line, a flexible member, a cable, a wire or another type of fastener known in the art may be used to provide fixation of the acetabular cup 7000 to a portion of bone.

At least one of the plurality of lobed fastener apertures 7020 may include a central aperture 7022 and an aperture lobe 7024. The central aperture 7022 may be configured to receive a central fastener 4100 and the aperture lobe 7024 may be configured to receive the locking fastener 4150. The aperture lobe 7024 may intersect with the central aperture 7022. The central aperture 7022 may be larger than the aperture lobes 7024.

The acetabular cup 7000 may include an articulating surface 7040 configured to receive a natural femoral head as part of a ball and socket joint. Additionally, or alternatively, the articulating surface 7040 may be configured to receive an anatomic prosthetic femoral head as part of a ball and socket joint. The acetabular cup 7000 may further include a bone engaging surface 7030 configured to be received in a pelvis 500 of a patient.

The plurality of lobed fastener apertures 7020 may extend from a first opening 7042 at the articulating surface 7040. The plurality of lobed fastener apertures 7020 may further include a second opening 7044 and a third opening 7046 at the bone engaging surface 7030. The first opening 7042 may include the central aperture 7022 and the aperture lobe 7024. The second opening 7044 may include the aperture lobe 7024. The third opening 7046 may include the central aperture 7022. The second opening 7044 and the third opening 7046 on the bone engaging surface 7030 may not intersect. The second opening 7044 and the third opening 7046 on the bone engaging surface 7030 may have a generally circular shape.

The first opening at the articulating surface 7040 may be larger than the second opening at the bone engaging surface 7030, thereby creating an aperture step 7026. The aperture step 7026 may be configured to engage a fastener head 4120 of the central fasteners 4100 and/or a fastener head 4170 of the locking fastener 4150, thereby allowing the central fastener 4100 and/or the locking fastener 4150 to secure the acetabular cup 7000 to the pelvis 500.

Additionally, or alternatively, the aperture lobe 7024 may include an aperture lobe threaded portion 7029. The fastener head 4170 of the locking fastener 4150 may include a proximal threaded portion 4180. The proximal threaded portion 4180 may correspond to the aperture lobe threaded portion 7029.

The aperture lobe threaded portion 7029 may be configured as a tapered thread whereby the tapered thread is larger at the articulating surface 7040 than at the bone engaging surface 7030. The proximal threaded portion 4180 may include a tapered thread that may be complementary to the tapered thread of the aperture lobe threaded portion 7029 thereby allowing the locking fastener 4150 to threadably engage the acetabular cup 7000 so that the fastener head 4170 is captive within the aperture lobe 7024 thereby allowing the locking fastener 4150 to secure the acetabular cup 7000 to the pelvis 500.

The central aperture 7022 and the fastener head 4120 of the central fastener 4100 may be configured so that the fastener head 4120 seats at a first depth within the lobed fastener apertures 7020. The aperture lobe 7024 and the fastener head 4170 of the locking fastener 4150 may be configured so that the fastener head 4170 seats at a second depth within the lobed fastener apertures 7020. The lobed fastener apertures 7020, the central fastener 4100, and the locking fastener 4150 may be configured so that the fastener head 4120 and the fastener head 4170 seat at generally the same depth within the lobed fastener apertures 7020.

The plurality of lobed fastener apertures 7020 may further be configured so that when a central fastener 4100 is received in the central aperture 7022 and anchored in the pelvis 500, the fastener head 4120 does not protrude from the lobed fastener aperture 7020 past the articulating surface 7040. The plurality of lobed fastener apertures 7020 may further be configured so that when a locking fastener 4150 is received in an aperture lobe 7024 and anchored in the pelvis 500, the fastener head 4170 does not protrude from the lobed fastener aperture 7020 past the articulating surface 7040.

When the central fastener 4100 is received in the central aperture 7022 and anchored in the pelvis 500, and the locking fastener 4150 is received in the aperture lobe 7024 and anchored in the pelvis 500, the locking fastener 4150 may be configured to inhibit the central fastener 4100 from disengaging from the pelvis 500. Additionally, or alternatively, a fastener head 4170 of at least one of the locking fasteners 4150 may overlap a fastener head 4120 of the central fastener 4100, thereby inhibiting the central fastener 4100 from disengaging from the pelvis 500.

Additionally, or alternatively, the plurality of lobed fastener apertures 7020, the central fastener 4100, and the locking fastener 4150 may be configured so that, with a threaded portion 4160 of the locking fastener 4150 anchored in the pelvis 500 and the threaded portion 4110 of the central fastener 4100 anchored in the pelvis 500, the fastener head 1170 of the locking fastener 1150 and the fastener head 1120 of the central fastener 1100 do not touch each other.

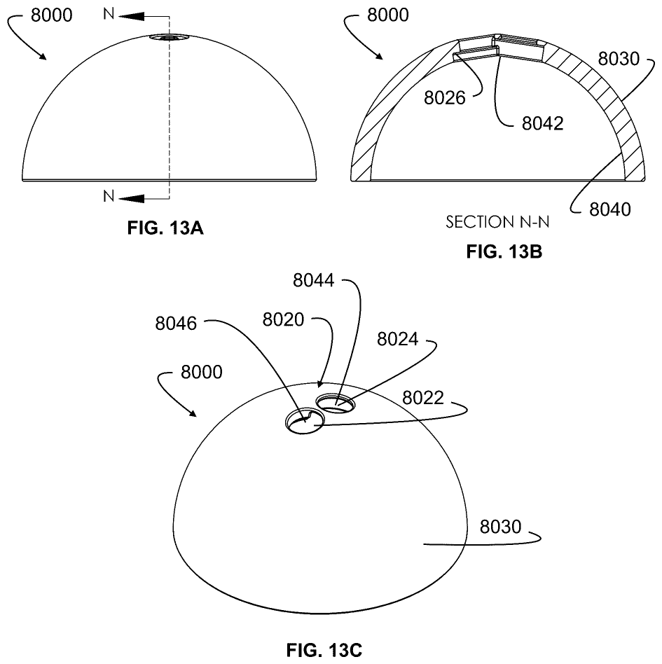

FIG. 13A is a side view of an acetabular cup 8000 according to an embodiment of the present disclosure. FIG. 13B is a front section view of the acetabular cup 8000 and FIG. 13C is a perspective view of the acetabular cup 8000. The acetabular cup 8000 may include similar function and features as the acetabular cup 1000. The acetabular cup 8000 may be configured to be received in a pelvis of a patient as part of a hip arthroplasty procedure. The acetabular cup 8000 may be configured to receive a natural femoral head as part of a ball and socket joint. Additionally, or alternatively, the acetabular cup 8000 may be configured to receive an anatomic prosthetic femoral head as part of a ball and socket joint.

An acetabular cup system may include an acetabular cup 8000, a first central fastener 1100, and a second central fastener 1100. Alternatively, an acetabular cup system may include an acetabular cup 2000 and a plurality of central fasteners 1100.

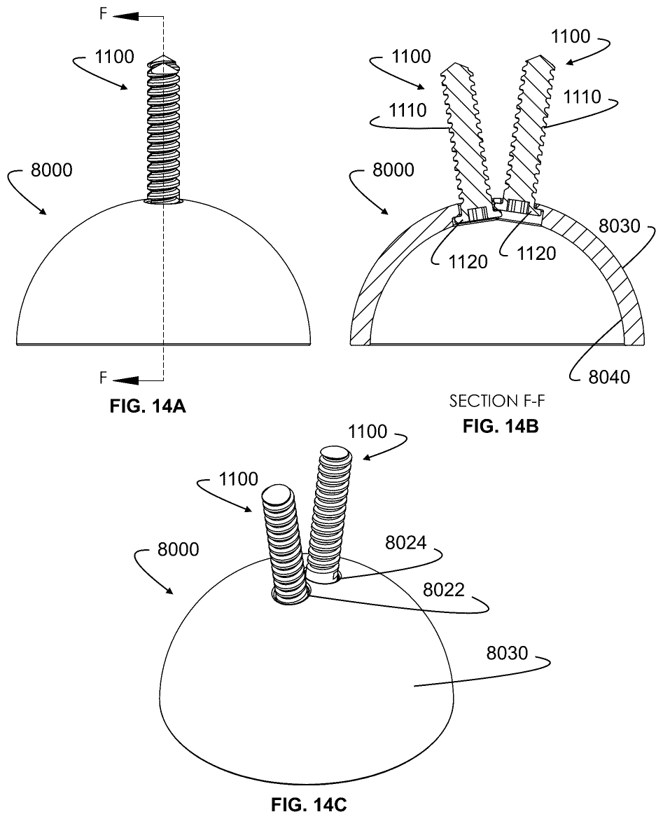

FIG. 14A is a side view of an acetabular cup 8000 and two central fasteners 1100 according to an embodiment of the present disclosure. FIG. 14B is a front section view of the acetabular cup 8000 and the two central fasteners 1100. FIG. 14C is a perspective view of the acetabular cup 8000 and the two central fasteners 1100. The acetabular cup 8000 may include a plurality of lobed fastener apertures 8020 configured to receive the two central fasteners 1100 within a single lobed fastener aperture 8020.

The central fastener 1100 may include a proximal end having a threaded portion 1110 and a distal end having a fastener head 1120. The plurality of lobed fastener apertures 8020 may be configured so that, with a first threaded portion 1110 anchored in the pelvis 500, and a second threaded portion 1110 anchored in the pelvis 500, a first fastener head 1120 and a second fastener head 1120 are adjacent, and the first threaded portion 1110 and the second threaded portion 1110 diverge within the pelvis 500.

The acetabular cup 8000 may include a plurality of lobed fastener apertures 8020. For clarity, an embodiment with a single lobed fastener aperture 8020 located at an apex of the acetabular cup 8000 is shown in FIGS. 14A-14C. In other embodiments, the acetabular cup 8000 may include two lobed fastener apertures 8020, three lobed fastener apertures 8020, four lobed fastener apertures 8020, five lobed fastener apertures 8020, six lobed fastener apertures 8020, or more than six lobed fastener apertures 8020.

For embodiments having two or more lobed fasteners apertures 8020, the lobed fastener apertures 8020 may be spaced equally around a bone engaging surface 8030 of the acetabular cup 8000. Additionally, or alternatively, one instance of the lobed fastener apertures 8020 may be located at the apex of the acetabular cup 8000. Additionally, or alternatively, the orientation of the lobed fastener apertures 8020 may differ at each instance along the acetabular cup 8000.

In an embodiment, the central fastener 1100 may be configured as a bone screw including a threaded portion 1110 and a fastener head 1120 and may be insertable through the lobed fastener apertures 8020 and may be configured to be anchored in a pelvis 500 to secure the acetabular cup 8000 to the pelvis 500. The threaded portion 1110 may include a cancellous thread form configured to engage a portion of cancellous bone. In other embodiments, the central fastener 1100 may be configured as a rivet, a brad, a nail, a bolt, a post, a peg, a staple, an anchor, a line, a flexible member, a cable, a wire or another type of fastener known in the art may be used to provide fixation of the acetabular cup 8000 to a portion of bone.

At least one of the plurality of lobed fastener apertures 8020 may include a central aperture 8022 and an aperture lobe 8024. The central aperture 8022 may be configured to receive a central fastener 1100 and the aperture lobe 8024 may also be configured to receive a central fastener 1100. The central aperture 8022 and the aperture lobe 8024 may be generally equally sized. The central aperture 8022 and the aperture lobe 8024 may have a generally circular shape.

The acetabular cup 8000 may include an articulating surface 8040 configured to receive a natural femoral head as part of a ball and socket joint. Additionally, or alternatively, the articulating surface 8040 may be configured to receive an anatomic prosthetic femoral head as part of a ball and socket joint. The acetabular cup 8000 may further include a bone engaging surface 8030 configured to be received in a pelvis 500 of a patient.

The plurality of lobed fastener apertures 8020 may extend from a first opening 8042 at the articulating surface 8040. The plurality of lobed fastener apertures 8020 may further include a second opening 8044, and a third opening 8046 at the bone engaging surface 8030. The first opening 8042 may include the central aperture 8022 and the aperture lobe 8024. The second opening 8044 may include an aperture lobe 8024. The third opening 8046 may include the central aperture 8022. The second opening 8044 and the third opening 8046 on the bone engaging surface 8030 may not intersect.

The first opening at the articulating surface 8040 may be larger than the second opening at the bone engaging surface 8030, thereby creating an aperture step 8026. The aperture step 8026 may be configured to engage a fastener head 1120 of the central fasteners 1100, thereby allowing the central fastener 1100 to secure the acetabular cup 8000 to the pelvis 500.

The central aperture 8022 and the first fastener head 1120 of the first central fastener 1100 may be configured so that the first fastener head 1120 seats at a first depth within the lobed fastener apertures 8020. The aperture lobe 8024 and the second fastener head 1120 of the second central fastener 1100 may be configured so that the second fastener head 1120 seats at a second depth within the lobed fastener apertures 8020. The lobed fastener apertures 8020 and the central fasteners 1100 may be shaped and configured such that the first fastener head 1120 and the second fastener head 1120 seat at different depths within the lobed fastener apertures 8020.

The plurality of lobed fastener apertures 8020 may further be configured so that when a first central fastener 1100 is received in the central aperture 8022 and anchored in the pelvis 500, the first fastener head 1120 does not protrude from the lobed fastener aperture 8020 past the articulating surface 8040. The plurality of lobed fastener apertures 8020 may further be configured so that when a second central fastener 1100 is received in an aperture lobe 8024 and anchored in the pelvis 500, the second fastener head 1120 does not protrude from the lobed fastener aperture 8020 past the articulating surface 8040.

When the first central fastener 1100 is received in the central aperture 8022 and anchored in the pelvis 500, and the second central fastener 1100 is received in the aperture lobe 8024 and anchored in the pelvis 500, the second central fastener 1100 may be configured to inhibit the first central fastener 1100 from disengaging from the pelvis 500. Additionally, or alternatively, a second fastener head 1170 of the second central fastener 1100 may overlap a first fastener head 1120 of the first central fastener 1100, thereby inhibiting the first central fastener 1100 from disengaging from the pelvis 500.

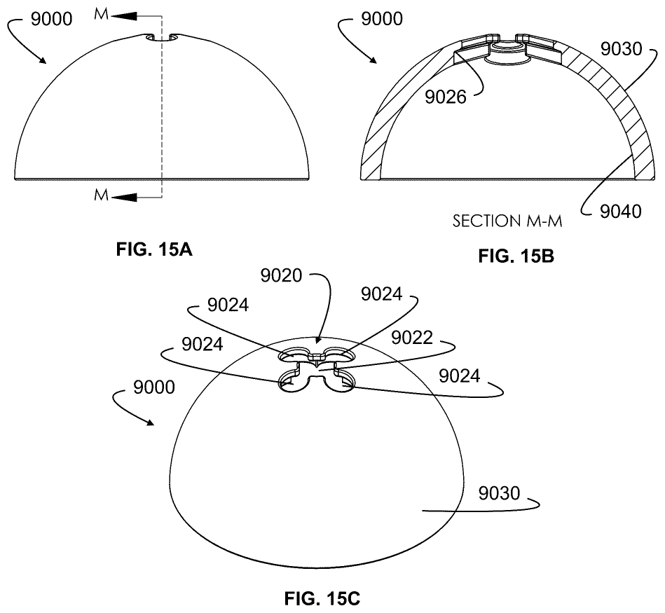

FIG. 15A is a side view of an acetabular cup 9000 according to an embodiment of the present disclosure. FIG. 15B is a front section view of the acetabular cup 9000 and FIG. 15C is a perspective view of the acetabular cup 9000. The acetabular cup 9000 may include similar function and features as the acetabular cup 1000. The acetabular cup 9000 may be configured to be received in a pelvis of a patient as part of a hip arthroplasty procedure. The acetabular cup 9000 may be configured to receive a natural femoral head as part of a ball and socket joint. Additionally, or alternatively, the acetabular cup 9000 may be configured to receive an anatomic prosthetic femoral head as part of a ball and socket joint.

An acetabular cup system may include an acetabular cup 9000, a central fastener 1100, and one or more locking fasteners 1150. The central fastener 1100 may be longer than the locking fasteners 1150. Alternatively, an acetabular cup system may include an acetabular cup 9000, a plurality of central fasteners 1100, and a plurality of locking fasteners 1150. Each of the plurality of central fasteners 1100 may be longer than the locking fasteners 1150.

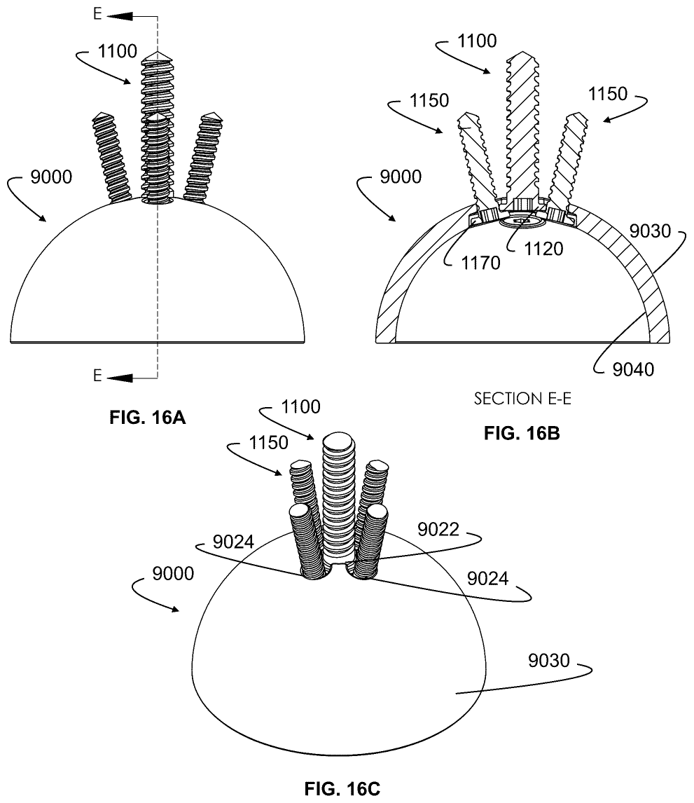

FIG. 16A is a side view of an acetabular cup 9000 a central fastener 1100, and four locking fasteners 1150 according to an embodiment of the present disclosure. FIG. 16B is a front section view of the acetabular cup 9000, the central fastener 1100, and the four locking fasteners 1150. FIG. 16C is a perspective view of the acetabular cup 9000, the central fastener 1100, and the four locking fasteners 1150. The acetabular cup 9000 may include a plurality of lobed fastener apertures 9020 configured to receive the central fastener 1100 and/or the locking fasteners 1150 within a single lobed fastener aperture 9020. At least one of the plurality of lobed fastener apertures 9020 may be configured to receive the central fastener 1100 and at least one of the locking fasteners 1150.

The central fastener 1100 may include a proximal end having a threaded portion 1110 and a distal end having a fastener head 1120. The locking fasteners 1150 may include a proximal end having a threaded portion 1160 and a distal end having a fastener head 1170. The plurality of lobed fastener apertures 9020 may be configured so that, with the threaded portion 1110 anchored in the pelvis 500, and the threaded portion 1160 anchored in the pelvis 500, the fastener head 1120 and the fastener head 1170 are adjacent, and the threaded portion 1110 and the threaded portion 1160 diverge within the pelvis 500.

Additionally, or alternatively, the plurality of lobed fastener apertures 9020 may be configured so that, with a threaded portion 1160 of a first of the locking fasteners 1150 anchored in the pelvis 500, and the threaded portion 1160 of a second of the locking fasteners 1150 anchored in the pelvis 500, the fastener head 1170 of the first of the locking fasteners 1150 and the fastener head 1170 of the second of the locking fasteners 1150 are adjacent, and the threaded portion 1160 of the first of the locking fasteners 1150 and the threaded portion 1160 of the second of the locking fasteners 1150 diverge within the pelvis 500.

The acetabular cup 9000 may include a plurality of lobed fastener apertures 9020. For clarity, an embodiment with a single lobed fastener aperture 9020 located at an apex of the acetabular cup 9000 is shown in FIGS. 16A-16C. In other embodiments, the acetabular cup 9000 may include two lobed fastener apertures 9020, three lobed fastener apertures 9020, four lobed fastener apertures 9020, five lobed fastener apertures 9020, six lobed fastener apertures 9020, or more than six lobed fastener apertures 9020.

For embodiments having two or more lobed fastener apertures 9020, the lobed fastener apertures 9020 may be spaced equally around a bone engaging surface 9030 of the acetabular cup 9000. Additionally, or alternatively, one instance of the lobed fastener apertures 9020 may be located at the apex of the acetabular cup 9000. Additionally, or alternatively, the orientation of the lobed fastener apertures 9020 may differ at each instance along the acetabular cup 9000.

In an embodiment, the central fastener 1100 may be configured as a bone screw including a threaded portion 1110 and a fastener head 1120 and may be insertable through the lobed fastener apertures 9020 and may be configured to be anchored in a pelvis 500 to secure the acetabular cup 9000 to the pelvis 500. The threaded portion 1110 may include a cancellous thread form configured to engage a portion of cancellous bone. In other embodiments, the central fastener 1100 may be configured as a rivet, a brad, a nail, a bolt, a post, a peg, a staple, an anchor, a line, a flexible member, a cable, a wire or another type of fastener known in the art may be used to provide fixation of the acetabular cup 9000 to a portion of bone.

In an embodiment, at least one of the locking fasteners 1150 may be configured as a bone screw including a threaded portion 1160 and a fastener head 1170 and may be insertable through the lobed fastener apertures 9020 and may be configured to be anchored in a pelvis 500 to secure the acetabular cup 9000 to the pelvis 500. The threaded portion 1160 may include a cancellous thread form configured to engage a portion of cancellous bone. In other embodiments, at least one of the locking fasteners 1150 may be configured as a rivet, a brad, a nail, a bolt, a post, a peg, a staple, an anchor, a line, a flexible member, a cable, a wire or another type of fastener known in the art may be used to provide fixation of the acetabular cup 9000 to a portion of bone.

At least one of the plurality of lobed fastener apertures 9020 may include a central aperture 9022 and four aperture lobes 9024. The central aperture 9022 may be configured to receive a central fastener 1100 and the four aperture lobes 9024 may be configured to receive at least one of the locking fasteners 1150. The four aperture lobes 9024 may intersect with the central aperture 9022. The central aperture 9022 may be larger than the aperture lobes 9024.

The acetabular cup 9000 may include an articulating surface 9040 configured to receive a natural femoral head as part of a ball and socket joint. Additionally, or alternatively, the articulating surface 9040 may be configured to receive an anatomic prosthetic femoral head as part of a ball and socket joint. The acetabular cup 9000 may further include a bone engaging surface 9030 configured to be received in a pelvis 500 of a patient.

The plurality of lobed fastener apertures 9020 may extend from a first opening at the articulating surface 9040 to a second opening at the bone engaging surface 9030. The first opening may include the central aperture 9022 and the four aperture lobes 9024. The second opening may also include the central aperture 9022 and the four aperture lobes 1024.

The first opening at the articulating surface 9040 may be larger than the second opening at the bone engaging surface 9030, thereby creating an aperture step 9026. The aperture step 9026 may be configured to engage a fastener head 1120 of the central fasteners 1100 and/or a fastener head 1170 of the locking fasteners 1150, thereby allowing the central fastener 1100 and/or the locking fasteners 1150 to secure the acetabular cup 9000 to the pelvis 500.

The central aperture 9022 and the fastener head 1120 of the central fastener 1100 may be configured so that the fastener head 1120 seats at a first depth within the lobed fastener apertures 9020. The aperture lobe 9024 and the fastener head 1170 of the locking fastener 1150 may be configured so that the fastener head 1170 seats at a second depth within the lobed fastener apertures 9020. The lobed fastener apertures 9020, the central fastener 1100, and the locking fastener 1150 may be shaped and configured such that the fastener head 1120 and the fastener head 1170 seat at different depths within the lobed fastener apertures 9020.

The plurality of lobed fastener apertures 9020 may further be configured so that when a central fastener 1100 is received in the central aperture 9022 and anchored in the pelvis 500, the fastener head 1120 does not protrude from the lobed fastener aperture 9020 past the articulating surface 9040. The plurality of lobed fastener apertures 9020 may further be configured so that when a locking fastener 1150 is received in an aperture lobe 9024 and anchored in the pelvis 500, the fastener head 1170 does not protrude from the lobed fastener aperture 9020 past the articulating surface 9040.

In an embodiment, the lobed fastener apertures 9020 may include one central aperture 9022 and four aperture lobes 9024. The central aperture 9022 may be configured to receive one central fastener 1100 and each of the four aperture lobes 9024 may be configured to receive a locking fastener 1150. The acetabular cup system may then include five bone fasteners and an acetabular cup 9000 having four aperture lobes 9024, wherein each of the four aperture lobes 9024 and the central aperture 1022 is configured to receive one of the five bone fasteners.

When the central fastener 1100 is received in the central aperture 9022 and anchored in the pelvis 500, and at least one locking fastener 1150 is received in at least one aperture lobe 1024 and anchored in the pelvis 500, the at least one of the locking fasteners 1150 may be configured to inhibit the central fastener 1100 from disengaging from the pelvis 500. Additionally, or alternatively, a fastener head 1170 of at least one of the locking fasteners 1150 may overlap a fastener head 1120 of the central fastener 1100, thereby inhibiting the central fastener 1100 from disengaging from the pelvis 500.

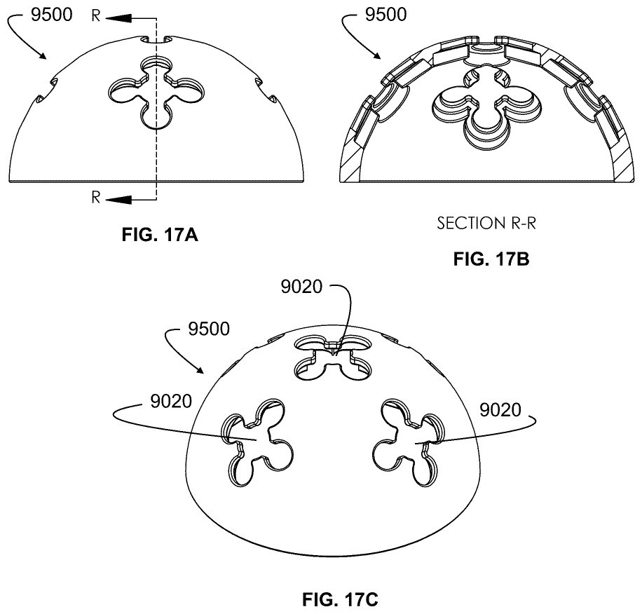

FIG. 17A is a side view of an acetabular cup 9500 according to an embodiment of the present disclosure. FIG. 17B is a front section view of the acetabular cup 9500 and FIG. 17C is a perspective view of the acetabular cup 9500. The acetabular cup 9500 may be an example of the acetabular cup 9000 with the plurality of lobed fastener apertures 9020 shown. Specifically, five lobed fastener apertures 9020 are shown evenly spaced on the body of acetabular cup 9500. Each of the plurality of lobed fastener apertures 9020 may include all features and functions previously described for lobed fastener apertures 9020 of acetabular cup 9000.

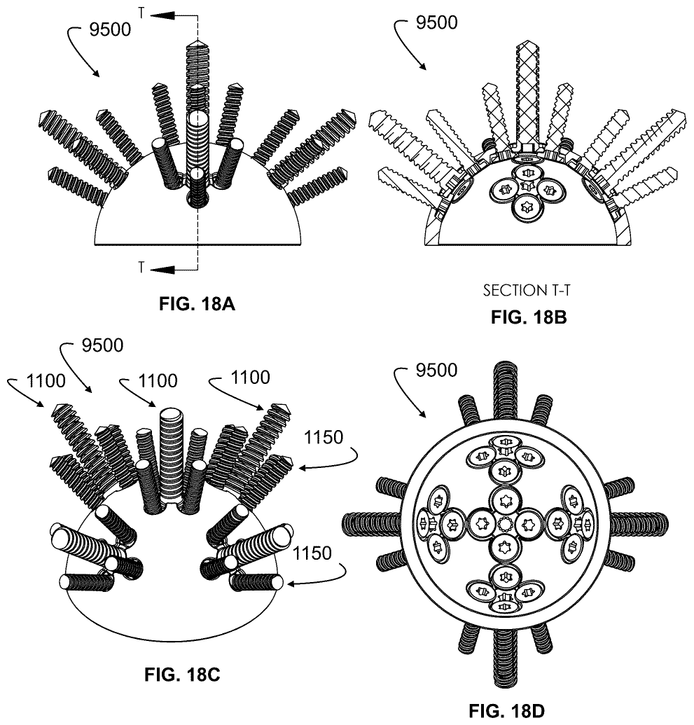

FIG. 18A is a side view of an acetabular cup 9500, five central fasteners 1100, and twenty locking fasteners 1150 according to an embodiment of the present disclosure. FIG. 18B is a front section view of the acetabular cup 9500, five central fasteners 1100, and twenty locking fasteners 1150. FIG. 18C is a perspective view of the acetabular cup 9500, five central fasteners 1100, and twenty locking fasteners 1150. FIG. 18D is a bottom view of the acetabular cup 9500, five central fasteners 1100, and twenty locking fasteners 1150. Each of the plurality of lobed fastener apertures 9020, central fasteners 1100, and locking fasteners 1150 may include all features and functions previously described for the acetabular cup system of FIGS. 16A-16C.

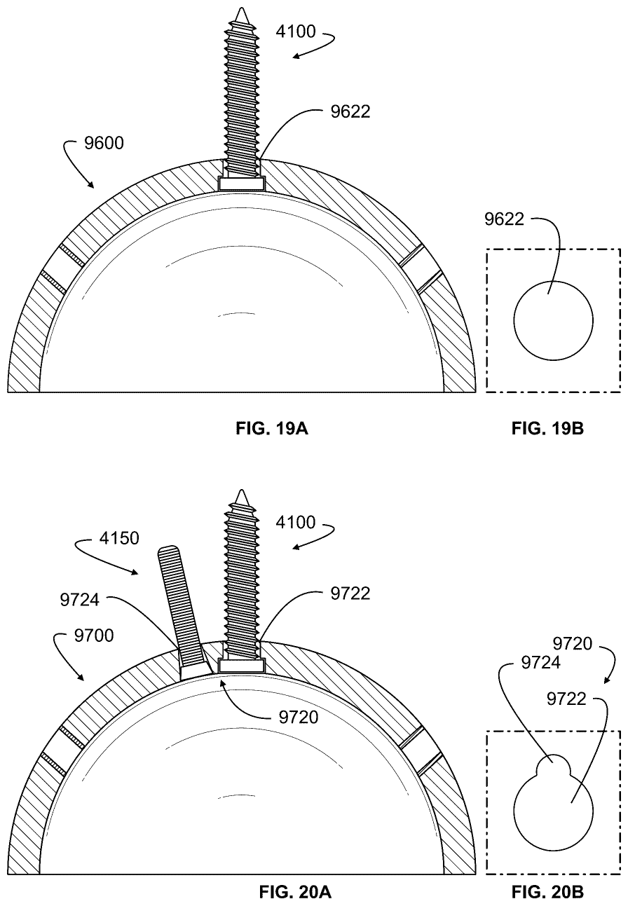

FIG. 19A is a front section view of an acetabular cup 9600 and a central fastener 4100 according to an embodiment of the present disclosure. FIG. 19B is a top detail view of a central aperture 9622 of the acetabular cup 9600. The acetabular cup 9600 may include similar features and function as the acetabular cup 5000. The acetabular cup 9600 may include a single central aperture 9622 configured to receive a central fastener 4100 to secure the acetabular cup 9600 to the pelvis.

FIG. 20A is a front section view of an acetabular cup 9700, a central fastener 4100, and a locking fastener 4150 according to an embodiment of the present disclosure. FIG. 20B is a top detail view of a lobed fastener aperture 9720 of the acetabular cup 9700. The acetabular cup 9700 may include similar features and function as the acetabular cup 8000. The acetabular cup 9700 may include a lobed fastener aperture 9720 having a central aperture 9722 configured to receive a central fastener 4100 and an aperture lobe 9724 configured to receive a locking fastener 4150.

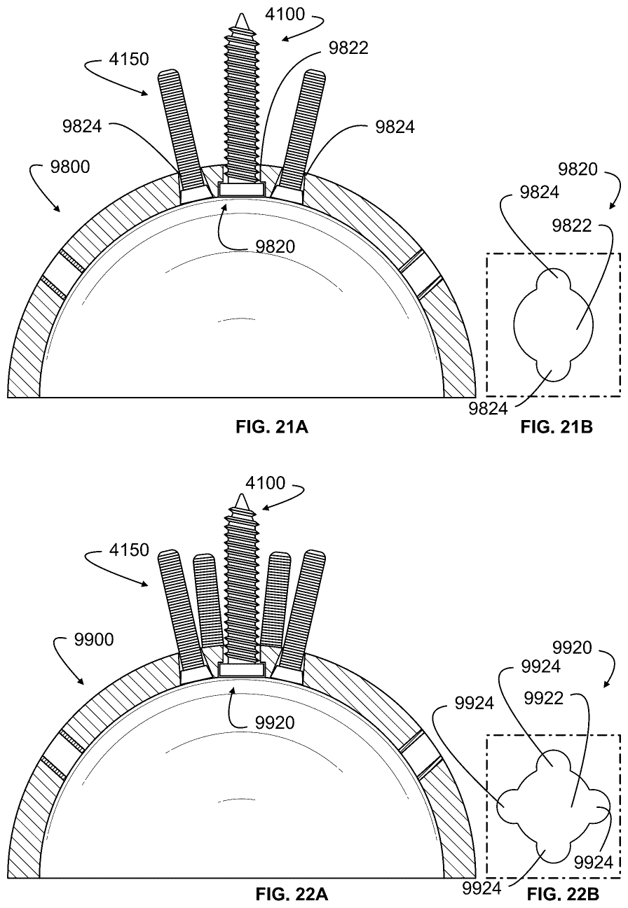

FIG. 21A is a front section view of an acetabular cup 9800, a central fastener 4100, and two locking fasteners 4150 according to an embodiment of the present disclosure. FIG. 21B is a top detail view of a lobed fastener aperture 9820 of the acetabular cup 9800. The acetabular cup 9800 may include similar features and function as the acetabular cup 2000. The acetabular cup 9800 may include a lobed fastener aperture 9820 having a central aperture 9822 configured to receive a central fastener 4100 and two aperture lobes 9824 configured to receive two locking fasteners 4150.

FIG. 22A is a front section view of an acetabular cup 9900, a central fastener 4100, and four locking fasteners 4150 according to an embodiment of the present disclosure. FIG. 22B is a top detail view of a lobed fastener aperture 9920 of the acetabular cup 9900. The acetabular cup 9900 may include similar features and function as the acetabular cup 9000. The acetabular cup 9900 may include a lobed fastener aperture 9920 having a central aperture 9922 configured to receive a central fastener 4100 and four aperture lobes 9924 configured to receive four locking fasteners 4150.

Acetabular cups with various configurations of lobed fastener apertures have been previously described. It is contemplated that a single acetabular cup may include a plurality of lobed fastener apertures wherein the plurality may include any combination of previously described lobed fastener apertures.

For example, an acetabular cup may include a four lobed fastener aperture at the apex of the acetabular cup and six two-lobed fastener apertures equally spaced around the body of the acetabular cup.

A second example may be, an acetabular cup may include a three lobed fastener aperture at the apex of the acetabular cup and four one-lobed fastener apertures equally spaced around the body of the acetabular cup.

A third example may be, an acetabular cup may include a two-lobed fastener aperture at the apex of the acetabular cup and four alternating no-lobed fastener apertures and four two-lobed fastener apertures equally spaced around the body of the acetabular cup.

The examples may show that the various iterations may be too numerous to explicitly describe, however, every possible acetabular cup that includes any combination of the lobed fastener apertures described within this disclosure should be considered within the spirit and scope of this disclosure.

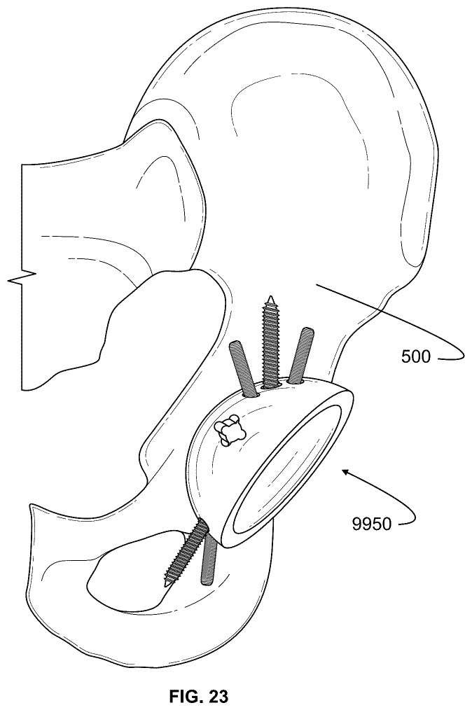

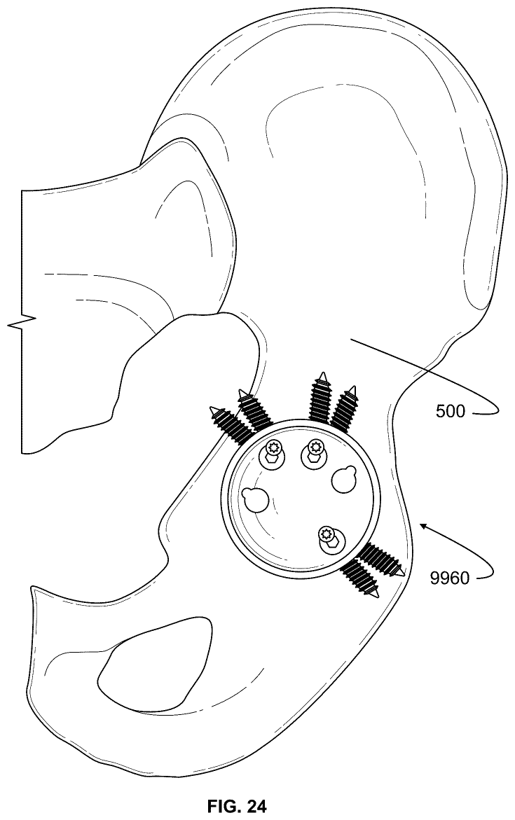

FIG. 23 is a perspective view of an acetabular cup 9950 with fasteners implanted in an exemplary pelvis 500 according to an embodiment of the present disclosure. FIG. 24 is a perspective view of an acetabular cup 9960 with fasteners implanted in an exemplary pelvis 500 according to an embodiment of the present disclosure.

An exemplary method for securing an acetabular cup system to a pelvis may include the following steps:
1. Preparing an acetabulum of a pelvis to receive an acetabular cup system.
2. Positioning the acetabular cup of the acetabular cup system proximate the prepared acetabulum.
3. Inserting one or more central fasteners through one or more lobed fastener apertures and anchoring the one or more central fasteners in the pelvis.
4. Inserting one or more locking fasteners through one or more lobe apertures and anchoring the one or more locking fasteners in the pelvis and thereby inhibiting the one or more central fasteners from disengaging from the pelvis.
5. Not all lobed fastener apertures may be used. The selection of lobed fastener apertures may be based on patient anatomy and/or bone quality.
6. Not all aperture lobes may be used for a given lobed fastener aperture. The selection of aperture lobes may be based on patient anatomy and/or bone quality.

Those of skill in the art will recognize that this is only one of many potential methods that may be used to secure an acetabular cup system to a pelvis. In alternative embodiments, different methods may be used to secure an acetabular cup system to a pelvis other than the methods described above. Further, the methods set forth above may be used to secure other acetabular cup systems besides those specifically disclosed herein.

Reference throughout this specification to “an embodiment” or “the embodiment” means that a particular feature, structure, or characteristic described in connection with that embodiment is included in at least one embodiment. Thus, the quoted phrases, or variations thereof, as recited throughout this specification are not necessarily all referring to the same embodiment.

Similarly, it should be appreciated that in the above description of embodiments, various features are sometimes grouped together in a single embodiment, figure, or description thereof for the purpose of streamlining the present disclosure. This method of disclosure, however, is not to be interpreted as reflecting an intention that any embodiment requires more features than those expressly recited in that embodiment. Rather, inventive aspects lie in a combination of fewer than all features of any single foregoing disclosed embodiment.

Recitation of the term “first” with respect to a feature or element does not necessarily imply the existence of a second or additional such feature or element. Elements recited in means-plus-function format are intended to be construed in accordance with 35 U.S.C. § 112 (f). It will be apparent to those having skill in the art that changes may be made to the details of the above-described embodiments without departing from the underlying principles set forth herein.

The phrases “connected to,” “coupled to,” “engaged with,” and “in communication with” refer to any form of interaction between two or more entities, including mechanical, electrical, magnetic, electromagnetic, fluid, and thermal interaction. Two components may be functionally coupled to each other even though they are not in direct contact with each other. The term “coupled” can include components that are coupled to each other via integral formation, as well as components that are removably and/or non-removably coupled with each other. The term “abutting” refers to items that may be in direct physical contact with each other, although the items may not necessarily be attached together. The phrase “fluid communication” refers to two or more features that are connected such that a fluid within one feature is able to pass into another feature. Moreover, as defined herein the term “substantially” means within +/−20% of a target value, measurement, or desired characteristic.

While specific embodiments and applications of the present disclosure have been illustrated and described, it is to be understood that the scope of this disclosure is not limited to the precise configuration and components disclosed herein. Various modifications, changes, and variations which will be apparent to those skilled in the art may be made in the arrangement, operation, and details of the devices, systems, instruments, and methods disclosed herein.

1. An acetabular cup system configured to be secured to a pelvis, the acetabular cup system comprising:
an acetabular cup comprising:
a bone engaging surface;
an articulating surface; and
a plurality of lobed fastener apertures extending from a first opening at the articulating surface to a second opening at the bone engaging surface; and
a plurality of bone fasteners insertable through the lobed fastener apertures to secure the acetabular cup to the pelvis;
wherein each of the plurality of lobed fastener apertures comprises:
a central aperture configured to receive a first bone fastener of the plurality of bone fasteners;
one or more aperture lobes intersecting with the central aperture configured to receive a second bone fastener of the plurality of bone fasteners; and
the central aperture is larger than the one or more aperture lobes.

2. The acetabular cup system of claim 1, wherein each of the lobed fastener apertures is configured so that, with the first bone fastener received in the central aperture and anchored in the pelvis, and the second bone fastener received in one of the one or more aperture lobes and anchored in the pelvis, the second bone fastener inhibits the first bone fastener from disengaging from the pelvis.

3. The acetabular cup system of claim 2, wherein the first bone fastener comprises a first fastener head, and the second bone fastener comprises a second fastener head, and wherein the lobed fastener apertures are shaped such that the first fastener head and the second fastener head seat at different depths within the lobed fastener apertures.

4. The acetabular cup system of claim 3, wherein the second fastener head overlaps the first fastener head.

5. The acetabular cup system of claim 1, wherein the first bone fastener is longer than the second bone fastener.

6. The acetabular cup system of claim 1, wherein the acetabular cup system comprises two aperture lobes and three bone fasteners, wherein each of the two aperture lobes and the central aperture is configured to receive one of the three bone fasteners.

7. The acetabular cup system of claim 1, wherein the first bone fastener comprises a first proximal end configured to engage the acetabular cup and a first distal end configured to be anchored within the pelvis, the second bone fastener comprises a second proximal end configured to engage the acetabular cup and a second distal end configured to be anchored within the pelvis, and the lobed fastener apertures are shaped such that the first proximal end and the second proximal end are adjacent and the first distal end and the second distal end diverge within the pelvis.

8. The acetabular cup system of claim 1, wherein the first bone fastener comprises a first fastener head comprising a first thread, the second bone fastener comprises a second fastener head comprising a second thread, and at least one of the central aperture and the one or more aperture lobes comprises a third thread, wherein the third thread corresponds to at least one of the first thread and the second thread.

9. An acetabular cup system, configured to be secured to a pelvis, the acetabular cup system comprising:
an acetabular cup comprising:
a bone engaging surface;
an articulating surface; and
a plurality of lobed fastener apertures extending from a first opening at the articulating surface to a second opening at the bone engaging surface; and
a plurality of bone fasteners insertable through the lobed fastener apertures to secure the acetabular cup to the pelvis;
wherein each of the plurality of fastener apertures comprises:
a first aperture configured to receive a first bone fastener of the plurality of bone fasteners;
a second aperture intersecting with the first aperture and configured to receive a second bone fastener of the plurality of bone fasteners; and
the first aperture and the second aperture are generally equally sized.

10. The acetabular cup system of claim 9, wherein the acetabular cup is further configured so that, with the first bone fastener received in the first aperture and anchored in the pelvis, and the second bone fastener received in the second aperture and anchored in the pelvis, the second bone fastener inhibits the first bone fastener from disengaging from the pelvis.

11. The acetabular cup system of claim 10, wherein the first bone fastener comprises a first fastener head, the second bone fastener comprises a second fastener head, and the fastener apertures are shaped such that the first fastener head and the second fastener head seat at different depths within the fastener apertures.

12. The acetabular cup system of claim 11, wherein the second fastener head overlaps the first fastener head.

13. The acetabular cup system of claim 9, wherein the first bone fastener comprises a first proximal end configured to engage the acetabular cup and a first distal end configured to be anchored within the pelvis, the second bone fastener comprises a second proximal end configured to engage the acetabular cup and a second distal end configured to be anchored within the pelvis, and the fastener apertures are shaped such that the first proximal end and the second proximal end are adjacent and the first distal end and the second distal end diverge within the pelvis.

14. The acetabular cup system of claim 9, wherein the first bone fastener comprises a first fastener head comprising a first thread, the second bone fastener comprises a second fastener head comprising a second thread, and at least one of the first aperture and the second aperture comprises a third thread, wherein the third thread corresponds to at least one of the first thread and the second thread.

15. An acetabular cup system configured to be secured to a pelvis, the acetabular cup system comprising:
an acetabular cup comprising:
a bone engaging surface;
an articulating surface; and
a plurality of lobed fastener apertures; and
a plurality of bone fasteners insertable through the lobed fastener apertures to secure the acetabular cup to the pelvis; and
wherein each of the plurality of fastener apertures comprises:
a central aperture extending from a first opening at the articulating surface to a second opening at the bone engaging surface and configured to receive a first bone fastener of the plurality of bone fasteners; and
at least one aperture lobe extending from a third opening at the articulating surface to a fourth opening at the bone engaging surface and configured to receive a second bone fastener of the plurality of bone fasteners;
wherein the first opening and the third opening intersect, and the second opening and the fourth opening do not intersect.

16. The acetabular cup system of claim 15, wherein the first bone fastener comprises a first fastener head, and the second bone fastener comprises a second fastener head, and wherein the acetabular cup is further configured so that, with the first bone fastener received in the central aperture and anchored in the pelvis, and the second bone fastener received in the aperture lobe and anchored in the pelvis, the first fastener head is not in contact with the second fastener head.

17. The acetabular cup system of claim 16, wherein the fastener apertures are shaped such that the first fastener head and the second fastener head seat at different depths within the fastener apertures.

18. The acetabular cup system of claim 15, wherein the first bone fastener is longer than the second bone fastener.

19. The acetabular cup system of claim 15, wherein the first bone fastener comprises a first proximal end configured to engage the acetabular cup and a first distal end configured to be anchored within the pelvis, the second bone fastener comprises a second proximal end configured to engage the acetabular cup and a second distal end configured to be anchored within the pelvis, and the fastener apertures are shaped such that the first proximal end and the second proximal end are adjacent and the first distal end and the second distal end diverge within the pelvis.

20. The acetabular cup system of claim 15, wherein the first bone fastener comprises a first fastener head comprising a first thread, the second bone fastener comprises a second fastener head comprising a second thread, and at least one of the central aperture and the aperture lobe comprises a third thread, wherein the third thread corresponds to at least one of the first thread and the second thread.
